(19) United States
(12) Patent Application Publication
(10) Pub. No.: US 2008/0274477 A1
(43) Pub. Date: Nov. 6, 2008

(54) Forms of Factor Xiia

(76) Inventor: David John Pritchard, Scone (GB)

Correspondence Address: Leon R Yankwich;Yankwich & Associates, 201 Broadway, Cambridge, MA, 02141, US

(21) Appl. No.: 11/795,074

(22) PCT Filed: Jan. 10, 2006
(86) PCT No.: PCT/GB2006/000072

§ 371 Date: May 27, 2008

(30) Foreign Application Priority Data
Jan. 11, 2005 (GB) 0500487.4

(51) Int. Cl. G01N 33/573; C12N 9/48; C12N 15/57; C07K 16/40; C12N 5/16; C12P 21/08; C12P 21/00

(52) U.S. Cl. 435/7.4; 435/212; 536/23.2; 530/387.9; 435/331; 435/70.21; 435/70.2

(57) ABSTRACT

A 53 Kd novel form of factor XIIa and related products, including nucleic acid molecules, monoclonal and polyclonal antibodies and hybridoma cell lines. Also assays for a 53 Kd form of factor XIIa and uses of said assays in diagnostic and prognostic methods, for example in the prediction of survival following myocardial infarction.

### INTRODUCTION

The present invention relates to Factor XIIa, a component of the “contact activation system”, and to a novel molecular weight form of Factor XIIa.

### BACKGROUND OF THE INVENTION

Factor XII is an inactive zymogen present in normal blood. It is readily converted, in vitro, in the presence of kallikrein, high molecular weight kininogen and a negatively charged surface into a form, Factor XIIa, that is enzymatically active. In vitro, two forms of XIIa have previously been reported. The 80 Kd form of the serine proteinase, often called Factor αXIIa, has a 52 Kd heavy chain linked by a disulphide bond to a 28 Kd light chain. Proteolysis of this factor releases a peptide from the heavy chain, and results in a product, Factor βXIIa, that retains serine protease activity, but in which the 28 Kd chain of Factor αXIIa is disulphide-linked to a small peptide fragment derived from the former 52-Kd heavy chain. In many cases the small peptide fragment has a molecular weight of about 1000 d, but fragments of different size have been observed in vitro.

WO90/08835 discloses an immunoassay for Factor XIIa. WO 90/08835 also discloses monoclonal antibodies 2/215 and 201/9, which bind to all known molecular weight forms of Factor XIIa, and methods for their production. Monoclonal antibody (mAb) 2/215 is produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606 and redeposited at ECACC on 14 Jun. 2004 under the deposit number 04061403. Hybridoma 201/9, producing monoclonal antibody 201/9, was deposited at ECACC on 18 Jan. 1990 under deposit number 90011893 and redeposited at ECACC on 14 Jun. 2004 under deposit number 04061402.

Factor XIIa has long been known to be involved in the contact system of blood coagulation in vivo. More recent work indicates that Factor XIIa is also involved in other systems, including fibrinolysis, kininogensis, and also complement activation and angiogenesis. Many clinical and experimental data are accumulating to suggest that the contact system extends beyond haemocoagulation and that it has a role in maintaining vascular wholeness and blood pressure, that it influences various functions of endothelial cells, and that it is involved in control of fibrinolysis and in maintaining the constitutive anticoagulant character of the intravascular space. Further clinical and experimental studies indicate that the contact system is involved in acute and chronic inflammation, shock of different aetiologies, diabetes, allergy, thrombo-haemorrhagic disorders including disseminated intravascular blood coagulation, and oncological diseases. Such conditions, include sepsis, spontaneous abortion and thromboembolism. In addition, Factor XIIa may be involved in tissue defence and repair. Yarovaya et al. (Yarovaya, G. A., Blokhina, T. B. & Neshkova, E. A. Contact system. New concepts on activation mechanisms and bioregulatory functions. Biochemistry (Mosc). 2002 January; 67(1): 13-24) is a recent review of the contact system and new concepts on activation mechanisms and bioregulatory functions.

WO 04/057343 discloses that Factor XIIa exists in a variety of forms in the body and that measurement of levels of those different forms provides valuable information relating to a variety of clinical conditions.

### SUMMARY OF THE INVENTION

The present invention provides a novel form of Factor XIIa having a molecular weight of 53 Kd as measured by High Performance Liquid Chromatography and mass spectroscopy. Preferably this novel form of Factor XIIa is human and has an amino acid sequence substantially as set out in FIGS. 1 and 2. The 53 Kd form of Factor XIIa has two peptide chains held together by a disulphide bridge. FIG. 1 shows the amino acid sequence of the first peptide chain, termed the “heavy chain”. FIG. 1 shows the amino acid sequence of the second peptide chain, termed the “light chain”. Preferably at least one of, more preferably both of the sequences differ by no more than 10%, still more preferably by no more than 8%, more preferably by no more than 6%, still more preferably by no more than 4%, still more preferably by no more than 2% from the sequence of FIGS. 1 and 2. Preferably, the heavy and light chains are of substantially the same length as set out in FIGS. 1 and 2. The present invention also provides an isolated nucleic acid molecule that encodes either or both of the peptides of the 53 Kd form of Factor XIIa.

The present invention provides an antibody that binds to one or more epitopes of the 53 Kd form of Factor XIIa and also provides an epitope-binding fragment or derivative of said antibody, for example a Fab fragment, a F(ab′)₂ fragment, a fragment produce by a Fab expression library or an anti-idiotypic (anti-Id) antibody, which has a corrected cross-reactivity with one or more of Factor αXIIa and Factor βXIIa of 10% or less, more preferably 5% or less, still more preferably 2% or less, still more preferably 1% or less, still more preferably 0.5% or less, still more preferably 0.1% or less. An antibody of the invention may be immobilized on a solid support or provided with a detectable label. An antibody of the invention may be a monoclonal antibody or a polyclonal antibody.

The invention also provides a hydridoma cell line that produces a monoclonal antibody of the invention and a method of producing such a monoclonal antibody by cultivating a hybridoma cell line of the invention in a growth medium and obtaining the antibody from the growth medium.

The invention also provides a method of producing polyclonal antibody serum comprising inoculating a mammal with an antigen present in the 53 Kd form of factor XIIa or an antigenic fragment thereof and purifying an antibody serum from the plasma of said mammal.

The invention also provides a method of producing a hydridoma cell line of the invention, comprising administering an antigen present in the 53 Kd form of factor XIIa to a mammal, obtaining antibody-producing cells from said mammal, fusing the resultant antibody-producing cells with a myeloma or otherwise immortalizing the cells, and screening the resultant hybridoma for the production of the monoclonal antibody.

The invention also provides a method of carrying out an immunoassay for an antigen in a sample of fluid, which assay comprises an interaction between an antigen and an antibody that binds thereto and the determination of the amount of antigen present in the sample with reference to results obtained using pre-determined amount of an antigen characterized in that the antibody is an antibody according to the invention and the antigen is a 53 Kd form of Factor XIIa.

The invention also provides a method of detecting and/or determining a 53 Kd form of Factor XIIa in a sample, which comprises subjecting the sample to a qualitative or quantitative immunoassay that comprises the interaction between an antigen and an antibody and the detection and/or determination of any resultant antigen-antibody complex, characterized in that the antibody is an antibody according to the invention and the antigen is a 53 Kd form of Factor XIIa.

The present invention provides a method for detecting or determining a 53 Kd form of Factor XIIa in a sample, which comprises carrying out a procedure that is capable of detecting or determining the 53 Kd form of Factor XIIa in preference to other forms of Factor XIIa, said other forms of Factor XIIa preferably being non-53 Kd forms of Factor XIIa.

In one embodiment, a method of the invention comprises detecting or determining a 53 Kd form of Factor XIIa under investigation by means of an assay that enables determination of a 53 Kd form of Factor XIIa under investigation in preference to other forms of Factor XIIa, said other forms of Factor XIIa preferably being non-53 Kd forms of Factor XIIa.

In another embodiment, a method of the invention comprises separating a 53 Kd form of Factor XIIa under investigation from other forms of Factor XIIa and detecting or determining the separated 53 Kd form of Factor XIIa, said other forms of Factor XIIa preferably being non-53 Kd forms of Factor XIIa.

The detection or determination of a separated 53 Kd form of Factor XIIa may be by means of an assay that enables determination of a 53 Kd form of Factor XIIa under investigation in preference to other forms of Factor XIIa, said other forms of Factor XIIa preferably being non-53 Kd forms of Factor XIIa.

In a further embodiment, a method of the invention comprises contacting the sample with a labeled antibody that is capable of binding to a 53 Kd form of Factor XIIa under investigation and that is optionally also capable of binding to other forms of Factor XIIa, separating the 53 Kd form of Factor XIIa under investigation from other forms, and detecting or determining the 53 Kd form of Factor XIIa, said other forms of Factor XIIa preferably being non-53 Kd forms of Factor XIIa.

The present invention also provides a method for diagnosing, monitoring, or predicting the susceptibility to, progress of, or outcome of a disease or disorder, or of treatment of the disease or disorder in a subject having or suspected of having the disease or disorder, which comprises detecting or determining a 53 Kd form of Factor XIIa in preference to other, preferably non-53 Kd forms of Factor XIIa in a sample obtained from the subject, and comparing the results obtained for the subject with the results obtained using the same assay for samples obtained from at least any one or more of the following:

(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

The present invention further provides a method comprising carrying out a series of assays for a 53 Kd form of Factor XIIa on samples obtained from subjects having a disease or disorder or treatment for a disease or disorder, and selecting an assay that provides information on levels of the 53 Kd form of Factor XIIa that is relevant to the disease or disorder or the treatment.

The present invention also provides a method for providing an assay for a 53 Kd form of Factor XIIa suitable for providing information relevant for diagnosing, monitoring, or predicting the susceptibility to, progress of, or outcome of a disease or disorder, or of treatment of the disease or disorder in a subject having or suspected of having the disease or disorder, which comprises carrying out a series of assays for the 53 Kd form of Factor XIIa on samples obtained from subjects having the disease or disorder or the treatment, and determining which assay(s) provide information on levels of the 53 Kd form of Factor XIIa that is relevant to diagnosing, monitoring, or predicting the susceptibility to, progress of, or outcome of the disease or disorder, or of treatment of the disease or disorder.

The method preferably comprises comprising comparing the results obtained for the 53 Kd form of Factor XIIa in the samples obtained from subjects having the disease or disorder or the treatment with the results obtained using the same assay for samples obtained from at least any one or more of the following:

(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

### BRIEF DESCRIPTION OF THE FIGURES

FIGS. 1 and 2 show the amino acid sequences of the two peptide chains that comprise 53 Kd XIIa. FIG. 1 shows the sequence of the “heavy” chain and FIG. 2 shows the sequence of the “light” chain.

FIG. 3 shows Factor XII zymogen in diagrammatic form. The thick dark line represents the peptide chain, disulphide bridges are shown by the thin and short lines. The amino terminus is labelled “N” and the carboxyl terminus is labelled “C”.

FIG. 4 shows Factor αXIIa in diagrammatic form using the same diagrammatic conventions as FIG. 3. It can be seen that compared to Factor XII a short region of the peptide chain is missing. The missing region cuts the remaining peptide into two chains, which remain held together by a disulphide bridge.

FIG. 5 shows Factor βXIIa in diagrammatic form using the same diagrammatic conventions as FIG. 3 and FIG. 4. It can be seen that, compared to Factor XII, the short region of the peptide chain missing in Factor αXIIa is also missing in Factor βXIIa. Additionally, a large portion of the amino end of the heavy chain of Factor αXIIa is missing.

FIG. 6 shows 53 Kd Factor XIIa in diagrammatic form using the same diagrammatic conventions as FIGS. 3 to 5. It can be seen that more of the heavy chain of Factor αXIIa is retained than is the case with Factor βXIIa, but that a shorter portion of the amino end of the heavy chain of Factor αXIIa is missing.

FIG. 7 shows traces resulting from HPLC separation of different forms of Factor XIIa bound to radioactively labelled monoclonal antibody 2/215 Fab. FIG. 7a shows the trace for molecular weight standards (670, 158, 44, 17, 1.35 kD). FIG. 7b shows the trace for radioactively labelled monoclonal antibody 2/215 Fab. FIG. 7c shows the trace for βXIIa plus radioactively labelled monoclonal antibody 2/215 Fab. FIG. 7d shows the trace for αXIIa plus radioactively labelled monoclonal antibody 2/215 Fab. FIG. 7e shows the trace for a typical plasma plus radioactively labelled monoclonal antibody 2/215 Fab.

FIG. 8 shows results of the mass spectroscopy experiment described in Example 2. The significant peaks at lower molecular masses (starred in the 201/9 plot) are multiple ionised species of the major 53 kD peak.

FIG. 9 shows results of the MALDI-TOF analysis following tryptic digest of the 53 Kd described in Example 3. The peaks in this plot represent the molecular masses of the resultant peptide sequences.

FIGS. 10 to 12 show data derived from the experiment described in Example 4.

FIG. 10 shows Kaplan Meier survival plots for patients admitted with chest pain. The patients are split into 4 quartiles on the basis of 53 kD XIIa concentration

FIG. 11 shows Kaplan Meier survival plots for patients admitted with chest pain and with Troponin T (TnT) greater than 0.05 ng/ml. The patients are split into 4 quartiles on the basis of 53 kD XIIa concentration

FIG. 12 shows Kaplan Meier survival plots for patients admitted with chest pain and with Troponin T (TnT) less than or equal to 0.05 ng/ml. The patients are split into 4 quartiles on the basis of 53 kD XIIa concentration

FIGS. 13 and 14 show data derived from the experiment described in Example 5.

FIG. 13 shows changes in the concentration of the 53 kD form of XIIa (expressed as pM) in patients over 4 days following admission to hospital with myocardial infarction.

FIG. 14 shows changes in the concentration of the 53 kD form of XIIa (expressed as percentage change from admission value) in patients over 4 days following admission to hospital with myocardial infarction.

### DEFINITIONS

Antibody includes any antibody fragment that is capable of binding antigen, for example, Fab and F(ab′)₂ fragments, and also recombinant, chimeric and humanized antibodies.

Antibody conjugate, also detection antibody, denotes an antibody labeled with a label that is directly or indirectly analyzable.

Capture antibody denotes an antibody that is immobilized on a solid phase for use in an immunoassay.

Capture assay denotes an immunoassay in which a capture antibody immobilized on a solid phase is contacted with a sample. If the sample comprises antigen capable of binding to the immobilized antibody and if the reaction conditions are appropriate, the antigen will form an antigen-antibody complex with the immobilized antigen and hence will be “captured” on the solid phase and can subsequently be detected or determined.

Cells, unless specified otherwise, denotes intact cells, cell remnants and cellular material.

Cellular Factor XIIa and cellular Factor XII denote Factor XIIa and Factor XII, respectively, present on the surface of a cell, or bound to a cell, cell remnants or cellular material.

Detection denotes a qualitative investigation.

Detection and/or determination denotes a quantitative or semi-quantitative investigation.

Factor XIIa, also called activated Factor XII, denotes any enzymatically active form or fragment of the zymogen, Factor XII.

High affinity binding partner denotes a molecule that forms a complex with Factor XIIa, which complex cannot be disrupted by simple methods, for example, by addition of a detergent or by competition with another species.

Lipid bound Factor XIIa denotes Factor XIIa associated with lipid material, for example, in association with lipids, especially lipoproteins and remnants thereof.

Low affinity binding partner denotes a molecule that forms a complex with Factor XIIa, which complex can be readily disrupted by simple methods, for example, by addition of a detergent or by competition with another species.

Monoclonal antibody (mAb) 2/215, also called antibody 2/215, is the antibody produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606, and redeposited at ECACC on 14 Jun. 2004 under the deposit number 04061403.

Monoclonal antibody (mAb) 2/215 analogue denotes an antibody that has Factor XIIa binding properties that are substantially the same as those of mAb 2/215.

Monoclonal antibody (mAb) 201/9, also called antibody 201/9, is the antibody produced by hybridoma 201/9, which was deposited at ECACC on 18 Jan. 1990 under deposit number 90012512 and redeposited at ECACC on 14 Jun. 2004 under the deposit number 04031402.

Monoclonal antibody (mAb) 201/9 analogue denotes an antibody that has Factor XIIa binding properties that are substantially the same as those of mAb 201/9.

Sample comprising cells denotes both samples of body fluids that comprise cells and samples of isolated cells.

Species and forms are terms that are used interchangeably in relation to Factor XIIa. They are used to distinguish between forms of Factor XIIa which are of different peptide length, what are termed “molecular weight variants” or “molecular weight forms” in this application, and also to distinguish between forms of Factor XIIa which are in different binding forms. The known molecular weight forms of Factor XIIa are Factor αXIIa, Factor βXIIa, Factor γXIIa and 53 Kd Factor XIIa. 53 Kd Factor XIIa is disclosed for the first time in this application. Examples of binding forms of Factor XIIa include cellular Factor XIIa, lipid-bound Factor XIIa and urinary Factor XIIa. Examples of “forms of Factor XIIa”, when that expression is used without further qualification include the following specific form: Cellular Factor αXIIa and Urinary 53 Kd Factor XIIa. The known non-53 Kd molecular weight forms of Factor XIIa are Factor αXIIa, Factor βXIIa and Factor γXIIa.

ug and ul denote micrograms and microlitres, respectively.

Urinary Factor XIIa denotes Factor XIIa present in urine.

### DETAILED DESCRIPTION OF THE INVENTION

### Molecular Weight Variants of Factor XIIa

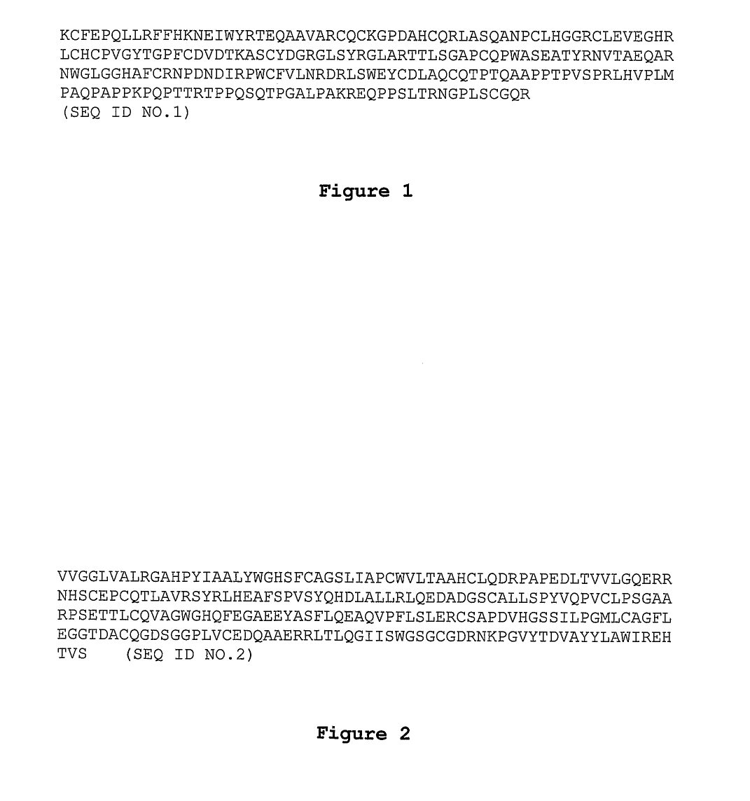

The present invention is based on our surprising observation that in vivo Factor XIIa (activated Factor XII) predominantly exists as a species with a molecular weight of approximately 53 Kd, and that measurement of this 53 Kd species is therefore likely to provide information relating a variety of clinical conditions. Such information will be more accurate than that which is derived from a method of measuring Factor XIIa that fails to discriminate between 53 Kd forms of Factor XIIa and other molecular weight forms of Factor XIIa. Although the novel 53 Kd forms of Factor XIIa around which this invention is based are conveniently termed “53 Kd forms of Factor XIIa”, or for short “53 Kd XIIa”, the term includes in its scope variants of Factor XIIa that have peptide chains of substantially the same lengths as 53 Kd XIIa, but that have a molecular weight that is different from 53 Kd due to alternative phosphorylation, glycosylation or other derivatisation, and also includes forms of 53 Kd XIIa that appear to have a non-53 Kd molecular weight when measured because they are complexed with or bound to other compounds. Therefore, the expression “a 53 Kd form of Factor XIIa” is used to indicate a form of Factor XIIa comprising a Factor XIIa peptide having similar peptide lengths to the archetypal 53 Kd form of Factor XIIa, regardless of the fact that reference to such forms of Factor XIIa includes reference to, for example, cellular 53 Kd XIIa, which may have a molecular weight substantially more than 53 Kd when measured whilst still complexed to a cellular component. “A 53 Kd form of Factor XIIa” or “a 53 Kd XIIa” refers to any form of Factor XIIa that comprises the novel 53 Kd molecular weight variant of Factor XIIa, for example urinary 53 Kd XIIa and cellular 53 Kd XIIa. Preferably the amino acid sequences of 53 Kd XIIa are similar to those disclosed in FIGS. 1 and 2. However, the term encompasses variants that are present in healthy individuals of a population but that vary in sequence because of naturally occurring allelic polymorphism and also artificially generated sequence variants that have been made by undertaking amino acid substitution, especially conservative amino acid substitutions, that do not materially affect the enzymatic activity or antigenicity of the peptide.

### Forms of Factor XIIa

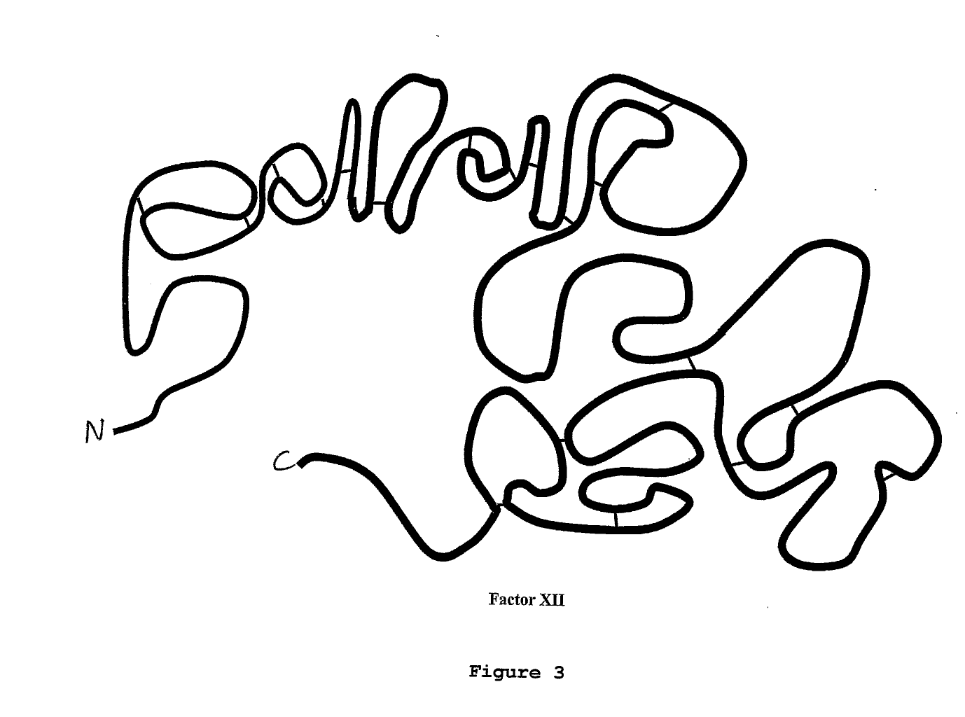

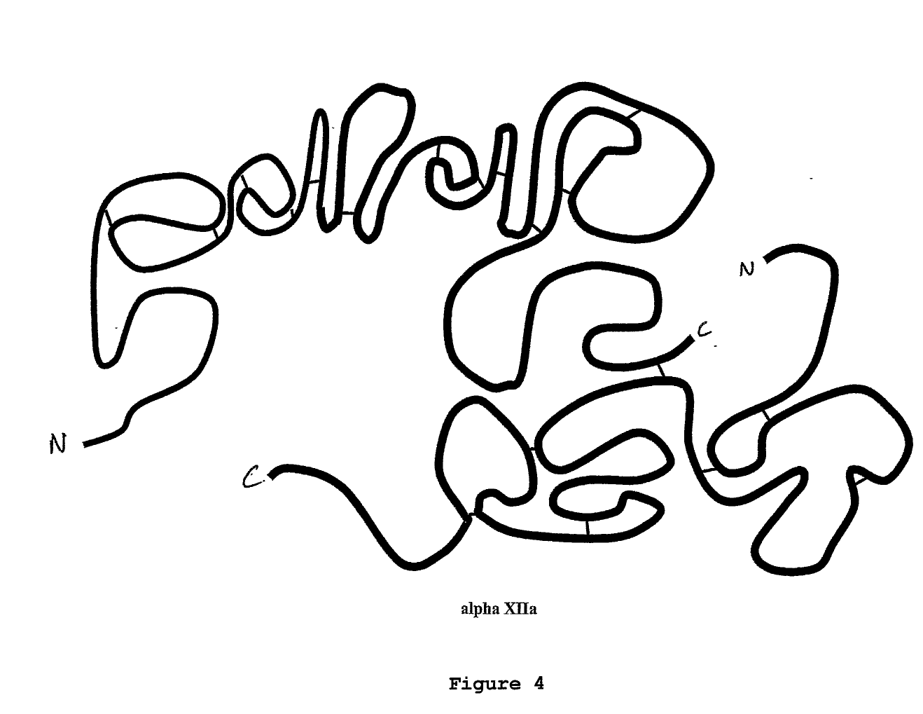

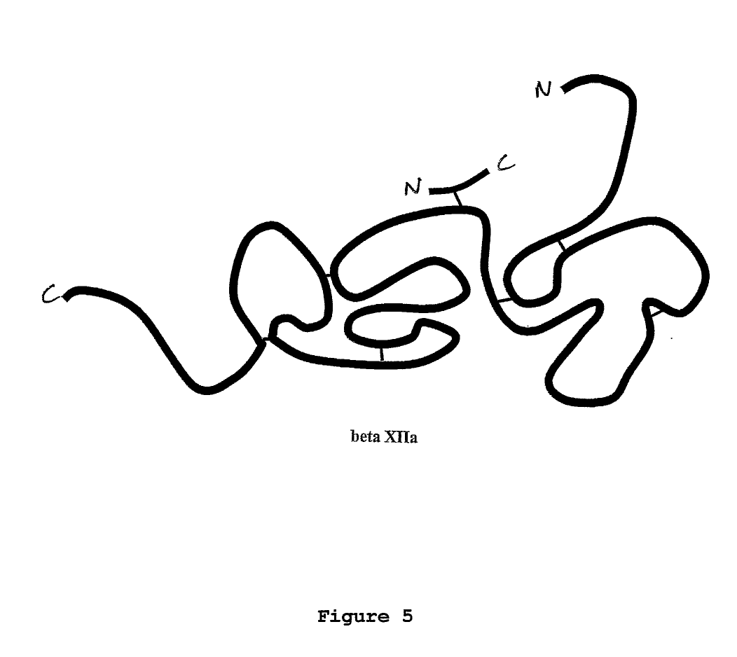

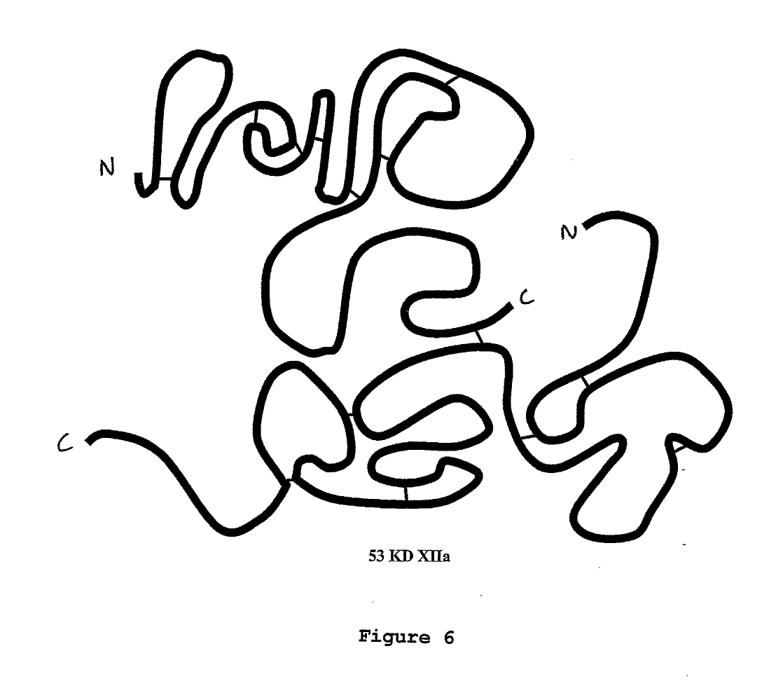

A diagrammatic representation of the molecular weight variants of the Factor XIIa peptide is shown in FIGS. 3 to 6. Variation in forms of Factor XIIa reflecting the molecular weight and peptide chain sequence of the Factor XIIa result from progressive cleavage of the inactive zymogen Factor XII shown in FIG. 3. Factor XII undergoes a cleavage resulting in an 80 Kd active serine proteinase, called Factor αXIIa and referred to as “alpha XIIa” in FIG. 4, that comprises a 52 Kd heavy chain linked by a disulphide bond to a 28 Kd light chain. Proteolysis of that Factor releases a peptide from the heavy chain and results in a product, called Factor βXIIa and referred to as “beta XIIa” in FIG. 5, that retains serine protease activity, but in which the 28 Kd chain of αXIIa is disulphide linked to a small peptide fragment derived from the former 52 Kd heavy chain. Factor βXIIa can undergo further proteolytic cleavage resulting in a fragment with a molecular weight of approximately 15 Kd, which has been called Factor γXIIa and is not illustrated in FIGS. 3 to 6. FIG. 6 shows the 53 Kd form of Factor XIIa, labelled as “53 KD XIIa”. As can be seen diagrammatically, 53 Kd XIIa shares the light chain of Factor αXIIa. That chain is coupled to a second chain that is shorter in length than that found in factor αXIIa, but longer than that found on Factor βXIIa. The amino acid sequences of the peptides of 53 Kd XIIa are given in FIGS. 1 and 2. For the avoidance of confusion, it should be noted that the nomenclature of “heavy chain” and “light chain” which derives from the relative sizes of the peptide chains found in Factor αXIIa (FIG. 4) is retained throughout this document when used to describe the peptide chains in other molecular weight forms of factor XIIa. Therefore the chain that corresponds to the light chain of Factor αXIIa continues to be referred to as the “light chain” when present in Factor βXIIa and 53 Kd Factor XIIa despite the fact that the other chain, which is referred to as the “heavy chain”, will in fact be the shorter chain in those proteins.

### 53 Kd Factor XIIa Proteins

53 Kd Factor XIIa proteins and their component peptide chains are included in the scope of the invention. Such proteins and peptides may be prepared for a variety of uses. Those uses include, but are not limited to, the generation of antibodies, use as reagents in diagnostic assays, and use as reagents in assays for screening for compounds that can be used as pharmaceutical agents for the treatment of medical disorders and diseases. The 53 Kd Factor protein sequences of the invention include the sequences presented in FIGS. 1 and 2 as well as analogues and derivatives thereof. Further, corresponding homologues from other species are encompassed by the invention. The invention also encompasses proteins that are functionally equivalent to 53 Kd Factor XIIa, such proteins include, but are not limited to, those containing substitutions of amino acid residues within the amino acid sequence of one or both of the peptides. Amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan and methionine; polar neutral amino acids include glycine, serine, threonine, cysteine, tyrosine, asparagines and glutamine; positively charged (basic) amino acids include arginine, lysine and histidine; and negatively charged (acidic) amino acids include aspartic acid and glutamic acid.

A variety of host-expression vector systems can be used to express the proteins and peptides of the invention. Details of suitable expression systems and methods of purification or enrichment of the proteins and peptides from the expression system may be found, for example in WO 02/00720, which is incorporated herein by reference.

### Nucleic Acids Encoding 53 Kd XIIa

The invention includes any nucleic acids capable of encoding a protein or peptide of the invention. Complements of those amino acids are also included in the scope of the invention. Nucleic acids of the invention may be isolated from a suitable nucleic acid library, may be isolated by PCR using suitable primers designed using the assistance of the sequences disclosed of Factor XII in GenBank Record NP_{—}000496, or synthesized by standard methods known in the art and described, for example, in WO 02/00720 and the references contained therein. Preferably nucleic acid of the invention solely encodes peptides of 53 Kd Factor XIIa. Preferably, nucleic acids of the invention do not contain sequence that encodes amino acid sequence that is absent from the peptides of 53 Kd Factor XIIa. Preferably, nucleic acids of the invention do not contain sequence that is absent from the peptides of 53 Kd Factor XIIa, but which is present in the peptide or peptides of one, more or all non-53 Kd form of Factor XIIa.

### Products Containing Nucleic Acids of the Invention

The invention also encompasses: DNA vectors, especially DNA expression vectors that contain one or more nucleic acid of the invention; genetically engineered host cells that contain one or more nucleic acid or DNA vector of the invention; and non-human transgenic animals, for example mice, rats, pigs, goats, cows or chickens that transgenicaly express a protein or peptide of the invention.

### Alternative Binding Forms of Factor XIIa

Factor XIIa in any one of its variant molecular weight forms, for example, as Factor αXIIa, βXIIa, γXIIa or 53 Kd XIIa can associate with other molecular species, including high affinity binding partners, for example, inhibitors, for example, C1 esterase inhibitor, and other binding proteins, for example, low affinity binding partners. It is postulated that association of Factor XIIa with such other binding proteins, for example, low affinity binding partners, may be reversible and may hinder binding to inhibitory proteins and hence reduce or prevent inhibition of Factor XIIa activity.

Factor XIIa in any one of its variant molecular weight forms, for example, as Factor αXIIa, βXIIa, γXIIa or 53 Kd XIIa may associate with and dissociate from lipids, for example, lipoproteins, which may be in the form of particles and/or remnants of particles. Factor XIIa in any one of its molecular weight variants forms, for example, as Factor αXIIa, βXIIa, γXIIa or 53 Kd XIIa may associate with and dissociate from any of cells and cellular fragments. Particularly in the case of Factor XIIa associated with cells, cellular fragments, lipoproteins and lipoprotein remnants, several molecules of a molecular weight form of Factor XIIa may be present on an individual particle. Furthermore, several molecules of Factor XIIa, either the same or different molecular weight forms, may be present as a complex of Factor XIIa molecules.

As was postulated in WO 04/057343 and illustrated in FIG. 1 of that application, it is thought that there exists in vivo a dynamic system of interconversions between various binding forms of Factor XIIa. It was also postulated that different binding forms of Factor XIIa and different molecular weight form of Factor XIIa have different roles in physiology and pathology, and that preferential measurement of particular forms of Factor XIIa will result in improved clinical utility in diagnosis, prediction and monitoring of diseases and disorders and treatment thereof, compared with measuring undefined forms of Factor XIIa.

### Cellular Factor XIIa

A number of authors have suggested that activation of Factor XII to Factor XIIa can occur on cell surfaces and have provided data to support that hypothesis. In particular authors have suggested that activation of Factor XII occurs on cells, notably endothelial cells, through the construction of multi-molecular assemblies that also contain High Molecular Weight Kininogen, Pre-kallikrein and Factor XI. These models indicate that, after it has been activated, Factor XIIa dissociates from the assembly and does not remain on the cell surface for a prolonged time, see for example, Yarovaya et al. (loc. cit.).

WO 04/057343 disclosed the observation that Factor XIIa exists in various binding forms, one of which is Factor XIIa present on the surface of cells circulating in the blood and on remnants thereof and on cellular material derived therefrom. This form of Factor XIIa was called “cellular Factor XIIa”.

A further observation was that, when Factor XIIa is cellular, not all Factor XIIa epitopes appear to be as accessible as when Factor XIIa is not cellular. For example, monoclonal antibody 2/215 is capable of binding effectively to cellular Factor XIIa and to non-cellular Factor XIIa. However, monoclonal antibody 201/9 and a sheep polyclonal antibody raised against Factor βXIIa do not appear to be able to bind as effectively to cellular Factor XIIa as to non-cellular Factor XIIa.

It appears that, in blood, Factor XIIa may be present in particular on granulocytes, especially a sub-population of granulocytes that, on flow cytometry, show a slightly higher scatter than other granulocytes, which indicates a different morphology from other sub-populations. These observations may have clinical implications.

### Lipid Bound Factor XIIa

It is also known from WO04/057343 that some Factor XIIa is associated with lipids, for example, lipoproteins and remnants thereof in the blood, and that measurement of this lipid bound Factor XIIa provides information relating to a variety of clinical conditions.

### Urinary Factor XIIa

WO 04/057343 also disclosed that Factor XIIa is present in urine, and that measurement of urinary Factor XIIa provides information relating to a variety of clinical conditions.

Molecular complexes and associations of Factor XIIa with other molecular species

Two or more molecules of Factor XIIa may be associated with each other in the form of a complex, also Factor XIIa may be associated with one or more other molecular species, for example, high affinity binding proteins, for example inhibitory molecules, or low affinity binding proteins. The results obtained when carrying out immunoassays in the presence and absence of a detergent, which would be expected to disrupt molecular complexes of Factor XIIa and associations of Factor XIIa with low affinity binding partners but not associations with high affinity binding partners, also indicate the presence of molecular complexes and associations with binding partners.

### Antibodies to Different Forms of Factor XIIa

Prior art antibodies exemplified by mAb 2/215 and mAb 201/9 do not bind to Factor XII, but bind to all known forms of Factor XIIa including the novel 53 Kd form of the invention. They cannot therefore be used to distinguish between different molecular weight forms of Factor XIIa unless they are used in conjunction with a technique that separates the molecular weight forms of Factor XIIa, either before of after binding of the antibody. Suitable methods of separating the molecular weight forms of Factor XIIa include chromatographic techniques, for example gel electrophoresis. An example of a specific assay for 53 Kd XIIa in which prior art antibodies may be used is an assay were prior art antibodies are used to immunoprecipitate Factor XIIa in general from a sample and the resulting Factor XIIa obtained is then run on a gel to separate the different molecular weight forms of Factor XIIa. The protein may then be visualised on the gel by staining with a general protein stain, or with one of the prior art antibodies, and the level of 53 Kd Factor XIIa may be determined by observing the intensity of the staining of the 53 Kd band on the gel.

Whilst an assay as described above is relatively easy to carry out, it would be desirable to simplify it still further by removing the need for a molecular weight separation. Such an improvement results in a single step assay and is therefore advantageous. The production of an antibody according to the present invention that is able to distinguish between 53 Kd Factor XIIa and one or more non-53 Kd forms of Factor XIIa is described below.

### Production of 53 Kd Factor XIIa-Specific Antibodies

For the production of antibodies, various host animals may be immunized by injection with a suitable antigen (see below for details of antigen selection). Such host animals may include but are not limited to pigs, rabbits, mice, goats, horses and rats. Various adjuvants may be used to increase the immunological response, depending on the host species, including but not limited to Freund's adjuvant (complete and incomplete), mineral salts such as aluminium hydroxide or aluminium phosphate, surface active substances such as lysolecithin, pluronic polyols, polyanions, peptides, oil emulsions, and potentially useful human adjuvants such as BCG (bacille Calmette-Guerin) and Corynebacterium parvum.

Alternatively, the immune response may be enhanced by combination and/or coupling with molecules of response-enhancing agents, for example, keyhole limpet haemocyanin, tetanus toxoid, diphtheria toxoid, ovalbumin, cholera toxin or fragments thereof.

Polyclonal antibodies are heterogeneous populations of antibody molecules derived from sera of the immunized animals.

Monoclonal antibodies, which are homogeneous populations of antibodies to a particular antigen, can be obtained by any technique that provides for the production of antibody molecules by continuous cell lines in culture.

These include, but are not limited to, the hybridoma technique of Kohler and Milstein, (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 7796).

A hybridoma that produces a mAb according to the present invention may be cultivated in vitro or in vivo and the resulting mAb purified by conventional techniques. Production of high titres of mAbs in vivo, may make this a preferred method of production. However, in vitro production may be preferred where legal, commercial or ethical constrains regarding the use of animals make in vivo production undesirable.

In addition, techniques developed for the production of “chimeric antibodies” (Morrison et al., 1984, Proc. Natl. Acad. Sci., 81: 6851-6855; Neuberger et al., 1984, Nature, 312: 604-608; Takeda et al., 1985, Nature, 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,075,181 and 5,877,397 and their respective disclosures which are herein incorporated by reference in their entirety. Also encompassed by the present invention is the use of fully humanized monoclonal antibodies as described in U.S. Pat. No. 6,150,584 which is herein incorporated by reference in their entirety. Human or humanised animal mAbs may be preferable for therapeutic use in humans.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 341: 544-546) can be adapted to produce single chain antibodies. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246: 1275-1281) to allow rapid and easy identification of monoclonal Fab fragments with the desired specificity.

Antibodies to 53 Kd Factor XIIa according to the invention can be utilized to generate anti-idiotype antibodies that “mimic” a 53 Kd Factor XIIa, using techniques known to those skilled in the art. (See, e.g., Greenspan & Bona, 1993, FASEB J. 7(5): 437-444; and Nissinoff, 1991, J. Immunol. 147(8): 2429-2438).

Antibodies of the invention may be of any immunoglobulin class including IgG, IgM, IgE, IgA, IgD and any subclass thereof.

### Selection and Preparation of Suitable Antigens for Production of Antibodies

### Antigen Selection

Antibodies according to the invention are required to bind to 53 Kd Factor XIIa in preference to at least one of Factor αXIIa and Factor βXIIa. They should therefore recognise an epitope or epitopes that are present and accessible on 53 Kd XIIa but are absent or inaccessible on at least one of Factor αXIIa and Factor βXIIa. One approach to antigen selection is therefore to select a peptide antigen having an amino acid sequence that is present on 53 Kd Factor XIIa but absent from Factor βXIIa. Such sequences may be found in the part of the heavy chain of 53 Kd Factor XIIa that is absent form Factor βXIIa. An alternative approach to antigen selection is to use substantially whole 53 Kd Factor XIIa or a peptide chain thereof or a fragment of either thereof with the aim of raising an antibody response against an epitope which incorporates the heavy chain N-terminus of 53 Kd Factor XIIa, this terminus being uniquely exposed in 53 Kd Factor. An alternative approach to antigen selection is to select as an antigen a peptide having a sequence which is present in Factor αXIIa and/or Factor βXIIa, but which is only exposed in 53 Kd Factor XIIa.

### Antigen Preparation

The size, extent of aggregation and relative nativity, that is to say, the relative lack or denaturation, of protein antigens can all dramatically affect the quality and quantity of antibody produced. Small polypeptides (<10 Kda) and non-protein antigens generally need to be conjugated or cross-linked to larger, immunogenic, carrier proteins to increase immunogenicity and provide T cell epitopes. Injection of soluble, non-aggregated proteins may induce tolerance rather than a satisfactory antibody response. It may therefore be desirable to conjugate the antigen to a larger protein such as keyhole limpet haemocyanin (KLH) or bovine serum albumen (BSA) Poly-L-lysine has also been used successfully as a backbone for small antigenic peptides.

The degree of nativity of the antigen may also need to be considered. Antibodies raised to native proteins react best with native proteins and antibodies to denatured proteins react best with denatured proteins. If antibodies are intended to be used to detect denatured proteins then the antibodies should preferably be raised against denatured antigens. On the other hand, if antibodies are to be used to detect native proteins, as is generally the case in diagnostic applications, antibodies should preferably be raised against native or substantially native antigens. The selection of an appropriate adjuvant may be used to alter the nativity of an antigen. Generally, absorbed protein antigens in a preformed oil-in-water emulsion adjuvant, retain greater native protein structure than those in water-in-oil emulsions.

Antigens should always be prepared using techniques that ensure that they are free of microbial contamination. Antigen preparations may be sterilized by passage through a 0.22 um filter.

### Purification of Polyclonal Antibodies

Polyclonal and monoclonal antibodies may be purified from non-immunoglobulin contaminants using known techniques, for example, use of a protein-A or protein-G affinity chromatography column. Polyclonal antibodies in accordance with the present invention may require further purification in order to eliminate or reduce cross reactivities. In order to remove cross-reactivities to non-53 Kd forms of Factor XIIa it may be necessary to remove those species of antibody from the polyclonal sera by a process of affinity purification. Fisher et al., 1988, Cell 54: 813-822, the disclosure of which is incorporated herein by reference, gives details of a suitable protocol for affinity purification of a polyclonal antibody. In essence, such purification techniques involves immobilizing the antigen or antigen that are causing the cross-reactivity problem on a solid substrate, for example the walls of an article of laboratory plastic-ware, or solid beads packed inside a chromatography column, and passing the polyclonal sera through or over the solid substrate so that antibody species exhibiting cross reactivity are retained and antibody species that do not show cross reactivity are retained in the liquid phase. As an example of the use of an affinity purification technique for the production of a polyclonal antibody of the present invention, an polyclonal antibody response could be raised in an animal by inoculating that animal with 53 Kd Factor XIIa; the resultant polyclonal sera could then be affinity purified by passing it through a chromatography column containing immobilised Factor αXIIa. Antibody species showing cross reactivity with Factor αXIIa would be retained in the column and antibody species capable of binding to 53 Kd Factor XIIa but not Factor αXIIa would remain in the liquid phase and be contained in the column eluate.

Detection and/or Determination of Different Forms of Factor XIIa

The present invention provides a method for detecting or determining a 53 Kd form of Factor XIIa, for example cellular 53 Kd XIIa, circulating 53 Kd XIIa or urinary 53 Kd XIIa, in a sample, which comprises carrying out a procedure that is capable of detecting or determining a 53 Kd form of Factor XIIa under investigation in preference to other molecular weight forms of Factor XIIa.

In one embodiment, a method of the invention comprises detecting or determining the 53 Kd form of Factor XIIa under investigation by means of an assay that enables determination of the 53 Kd form of Factor XIIa under investigation in preference to other forms of Factor XIIa, said other forms of Factor XIIa preferably being non-53 Kd forms of Factor XIIa.

In another embodiment, a method of the invention comprises separating a 53 Kd form of Factor XIIa from other forms of Factor XIIa and detecting or determining the separated 53 kd form of Factor XIIa, said other forms of Factor XIIa preferably being non-53 Kd forms of Factor XIIa.

The detection or determination of the separated 53 Kd form of Factor XIIa may be by means of an assay that enables determination of the 53 kd form of Factor XIIa in preference to other forms of Factor XIIa, said other forms of Factor XIIa preferably being non-53 Kd forms of Factor XIIa.

In a further embodiment, a method of the invention comprises contacting the sample with a labeled antibody that is capable of binding to a 53 kd form of Factor XIIa and that is optionally also capable of binding to other forms of Factor XIIa, separating the 53 kd form of Factor XIIa from other forms of Factor XIIa, said other forms of Factor preferably being non-53 Kd forms of factor XIIa, and detecting or determining the 53 Kd form of Factor XIIa.

According to the invention, therefore, a 53 Kd form of Factor XIIa may first be separated from other forms of Factor XIIa, preferably other molecular weight forms of Factor XIIa, and then the 53 Kd form of Factor XIIa may be determined. A general assay for Factor XIIa may be used i.e. an assay that is not specific for any particular form of Factor XIIa, but it may be advantageous to use an assay that enables determination of the 53 Kd form of Factor XIIa in preference to other forms of Factor XIIa, said other forms of Factor XIIa preferably being non-53 Kd forms of Factor XIIa. Examples of such assays are given below. Such a procedure may be used to detect or determine, for example, cellular 53 Kd XIIa, molecular complexes and associations of 53 Kd XIIa with other molecular species.

Alternatively, an assay that enables determination of a 53 Kd form of Factor XIIa under investigation in preference to other forms of Factor XIIa, preferably other non-53 Kd forms of Factor XIIa, may be carried out directly on a sample without previous separation of different forms of Factor XIIa. Examples of such assays are given below. Such an assay may be carried out directly on a sample. Such a procedure may be used to detect or determine, for example, molecular complexes and associations of Factor XIIa with other molecular species.

As a further alternative, a sample comprising 53 Kd forms of Factor XIIa may be contacted with a labeled antibody and then separation of the molecular weight forms of Factor XIIa under investigation may be carried out, with detection or determination of the separated forms. Such a procedure may be used to detect or determine, for example, lipid bound 53 Kd Factor XIIa.

### Separation of Forms of Factor XIIa

Forms of Factor XIIa may be separated on the basis of their physical, chemical or immunological properties. Any such separation should generally be carried under conditions such that the form or forms of Factor XIIa under investigation are maintained unchanged, for example, the conditions should generally be such that any complexes or molecular associations are not disrupted, and that any form of Factor XIIa bound to another material, for example, to cellular or lipid material, is not released from that material. However, in some circumstances it may be desired to release Factor XIIa from an association or from material to which it is bound.

### Separation on the Basis of Physical Properties

Different molecular weight forms of Factor XIIa may be separated on the basis of molecular weight, for example, using chromatographic procedures, for example, High Pressure Liquid Chromatography (HPLC), flow cytometry or ultracentrifugation techniques, followed by assessment of the separated material.

Assessment can be done in several ways, for example by use of an immunoassay on the separated forms, or by use of an enzymatic assay, for example using a chromogenic substrate such as S2302 (Kabi Diagnostics, Uxbridge, England). Antibodies against Factor XIIa may be used in conjunction with HPLC. For example, labeled antibodies may be reacted with the sample, and the resulting mixture may be subjected to HPLC separation. The complexes of antibody with particular molecular weight forms of Factor XIIa can then be determined using a suitable detection system for the material used to label the antibody.

Separation of Molecular Complexes and of Associations of Factor XIIa with Binding Partners on the Basis of Physical Properties

Such a method may be useful, inter alia, for separating molecular complexes comprising two or more molecules of Factor XIIa from other forms of Factor XIIa, and also for separating forms of Factor XIIa associated with high affinity or low affinity binding partners.

It is generally preferable to carry out such separation under conditions such that Factor XIIa complexes are not disrupted and that Factor XIIa is not dissociated from a binding partner. For example, it is generally preferable to avoid the presence of detergents, which tend to disrupt complexes and some molecular associations. However, in some circumstances it may be desirable that disruption occurs. For example, if it is desired to release Factor XIIa from low affinity binding partners or to separate Factor XIIa associated with low affinity binding partners from Factor XIIa associated with high affinity binding partners, appropriate conditions, for example, a detergent, may be used, resulting in dissociation of Factor XIIa from low affinity binding partners but not from high affinity binding partners.

### Separation of Cellular Factor XIIa and Lipid Bound Factor XIIa on the Basis of Physical or Chemical Properties

Cellular and lipid bound Factor XIIa may be separated from other forms of Factor XIIa by physical or chemical methods, or by combinations thereof. For example, cellular Factor XIIa may be separated by centrifugation or flow cytometry. Lipid bound Factor XIIa may be separated, for example, by lipoprotein precipitation agents and, generally, centrifugation, or by density layer ultracentrifugation.

It is generally preferable to carry out separation under conditions such that the Factor XIIa is not dissociated from the cellular or lipid material. For example, it is generally preferable to avoid the presence of detergents. However, in some circumstances it may be desirable that disruption occurs. If it is desired to separate Factor XIIa from the material to which it is bound, appropriate conditions may be used.

### Immunological Separation

A form or forms of Factor XIIa under investigation may be separated from other forms by means of an immunological method using antibodies that show preferential binding for the form or forms of Factor XIIa under investigation. For example, immunoaffinity chromatography may be carried out, the antibody being immobilized on an appropriate solid support. Measurement of enzymic activity in either the bound or un-bound fractions may be carried out after chromatography. Preferred antibodies are those which recognize one or more epitopes of a 53 Kd form of Factor XIIa and which have corrected cross reactivity with one or both of Factor αXIIa and Factor βXIIa of 0.1% or less. The production of such antibodies is described elsewhere in this specification.

As described above in relation to separation on the basis of physical or chemical properties, separation by immunoaffinity chromatography should generally be carried out under conditions such that the form or forms of Factor XIIa is/are maintained unchanged, for example, complexes and associations are not disrupted and bound molecules are not released. However, there may be circumstances when disruption is desired. If so, appropriate conditions may be used. Immunoaffinity chromatography is preferably carried out using an antibody that recognizes one or more epitopes of a 53 Kd form of Factor XIIa but which has corrected cross reactivity with one or both of Factor αXIIa and Factor βXIIa of 10% or less, more preferably 5% or less, still more preferably 2% or less, still more preferably 1% or less, still more preferably 0.5% or less, still more preferably 0.1% or less. The production of such antibodies is described elsewhere in this specification.

### Determining Suitability of Assays

Methods for detecting or determining Factor XIIa without discrimination as to molecular weight form are known and include chromogenic, for example, amidolytic assays and various types of immunoassays, for example, immunoassays using prior art antibodies.

If the 53 Kd form or forms of Factor XIIa under investigation have been separated from other molecular weight forms of Factor XIIa before the assay is carried out, an assay that does not discriminate between different molecular weight forms of Factor XIIa may be used i.e. a “general” Factor XIIa assay. Even after a prior separation step it may, however, be advantageous to use an assay that is capable of detecting or determining the 53 Kd form or forms of Factor XIIa under investigation preferentially in relation to other molecular weight forms.

If no separation step is carried out, the assay used must be capable of detecting or determining the 53 Kd form or forms of Factor XIIa under investigation from other non-53 Kd forms of Factor XIIa. An assay known to suitable for detecting or determining Factor XIIa may be tested for the ability to detect or determine the desired form or forms of 53 Kd Factor XIIa in a sample.

For example, using a sample known to comprise cellular 53 Kd Factor XIIa, the results obtained for an assay under investigation are compared with the results obtained using an assay known to be suitable for the detection of cellular 53 Kd Factor XIIa. Monoclonal antibody 2/215 is capable of binding effectively to cellular 53 Kd Factor XIIa. An immunoassay involving mAb 2/215 or an analogue thereof may be used as a comparison assay. The same considerations apply to other forms of 53 Kd Factor XIIa.

An alternative is to carry out the assay under investigation on a portion of a sample known to comprise the desired form of 53 Kd Factor XIIa, for example, cellular 53 Kd Factor XIIa. In that case, the sample should not contain non-cellular 53 Kd Factor XIIa. Another portion of the sample is treated to release the 53 Kd Factor XIIa from the cells, the treated cells are isolated, the assay is repeated, and the results of the two assays are compared. If result obtained for the assay on the sample that contains cellular 53 Kd Factor XIIa is higher than that obtained from the sample treated to remove the cellular 53 Kd Factor XIIa, that indicates that the assay is suitable for detecting or determining cellular 53 Kd Factor XIIa. The same considerations apply to other forms of 53 Kd Factor XIIa.

### Specificity of an Assay for One or More Forms of Factor XIIa

Specificity of an assay for one or more forms of Factor XIIa relative to other forms may be achieved or improved by design of the assay. The parameters of the assay may be adjusted such that the forms or forms of Factor XIIa under investigation is/are detected or determined preferentially relative to other forms of Factor XIIa.

Such optimization of an assay is standard practice in the art, and suitable techniques are well known, see for example, Principles and Practice of Immunoassays, Eds. Price C P & Newman D J, Stockton Press, 1991.

In the case of an immunoassay, parameters that can be adjusted to achieve a desired specificity may include any one or more of choice of the antibody or combination of antibodies to be used; presence, absence and choice of a detergent; and conditions used for plate coating in the case of an antigen capture assay involving an antibody coated on a solid phase.

For example, in the case of microtitre plate immunoassays there are a number of parameters that may be altered to measure certain forms of Factor XIIa preferentially relative to other forms.

The formulation of the solution used for coating the solid phase with capture antibody also affects the preferential measurement of different forms of Factor XIIa, for example, the concentration of antibody included in the formulation, and the pH and constituents of the buffer are important.

A further parameter that influences which forms are preferentially measured is the presence or absence of a detergent, for example, Triton, in the sample during incubation with the antibody. It is postulated that the presence of a detergent may disrupt complexes, for example, complexes of Factor XIIa molecules, and/or may release Factor XIIa previously bound to cells and/or lipids. The nature and/or amount of a detergent may also influence the assay.

An additional example of a parameter that can be manipulated to affect the preferential measurement of particular forms of Factor XIIa is the choice of antibody that is labelled to form the conjugate used for detecting antigen-antibody complexes.

It should be noted that there are complex interactions between the assay parameters, for example the effect of incorporating a detergent in an assay is dependent upon the combination of capture antibody, coating antibody concentration, coating buffer, and conjugate antibody used. The optimum conditions for detecting or determining a desired form of Factor XIIa may be determined by appropriate manipulation of the various parameters, in accordance with normal practice in the art.

If an assay is intended to discriminate between 53 Kd forms of Factor XIIa and non-53 Kd forms of Factor XIIa by use of an antibody that recognises epitopes uniquely present on 53 Kd forms of Factor XIIa, care must be taken in the design of the assay conditions to ensure that the integrity of the epitopes of interest are available for antibody binding. Certain epitopes may only be available if the 53 Kd Factor XIIa is non-denatured. Other epitopes may require denaturation of one or more form of Factor XIIa in order to be revealed. Choice of assay conditions will influence the extend to protein denaturation. For example, use of detergent or high ion concentrations will generally result in increased levels of denaturation. Carrying out and assay and/or sample preparation stage in under reducing conditions (for example in the presence of a reducing agent such as mercaptoethanol) may result in the separation of the heavy and light chains of Factor XIIa. Depending on the epitopes used in the assay, this may lead to the destruction of epitopes or alternatively the exposure of hitherto hidden epitopes.

### Samples and Sample Preparation

### Samples

Measurement of different forms of 53 Kd Factor XIIa may be performed on a sample of a body fluid, for example, whole blood, plasma, serum, urine, cerebrospinal fluid, saliva or tears; or a sample comprising cells isolated from a body fluid, that is to say, cells substantially free from the liquid phase in which they exist in vivo; or a sample comprising tissue or cells obtained from a tissue sample.

### Sample Preparation

Samples may be obtained and prepared according to normal practice, see for example, Young, D. S. & Bermes, E. W. “Specimen collection and processing” in Tietz Textbook of Clinical Chemistry 2^{nd} Edition” Eds. Burtis, C. A. & Ashwood, E. R., Saunders (1994), also Methods in Enzymology, H. Van Vunakis and J. J. Langone (Eds), 1981, 72(B); Practice and Theory of Enzyme Immunoassays, P Tijssen, Laboratory Techniques in Biochemistry and Molecular Biology, R. J. Burden and P. H. Van Knippenberg (Eds), Elsevier, 1985; Introduction to Radioimmunoassay and Related Techniques, T. Chard, ibid, 3rd Edition, 1987; and Methods in Enzymology, H. Van Vunakis and J. J. Langone (Eds) 1981, 74(C).

### Body Fluids

According to the present invention, one or more 53 Kd forms of Factor XIIa may be detected or determined in a sample of a body fluid. Examples of body fluids are whole blood, plasma, serum, urine, cerebrospinal fluid, saliva and tears. Samples of body fluid may be obtained and prepared in a conventional manner, for example, as described in the references above.

The selective measurement of particular forms of Factor XIIa in preference to other forms may be achieved as in the section on assays below.

### Cellular Factor XIIa

In one embodiment, the present invention provides a method which comprises detecting or determining 53 Kd Factor XIIa in a sample comprising cells obtained from a mammalian subject, generally a human, particularly cells circulating in blood or another body fluid.

Measurement of cellular Factor XIIa may be performed on a sample of a body fluid, or cells may be isolated, that is to say, made substantially free from the liquid phase in which they exist in vivo, from a sample of a body fluid, for example, whole blood or plasma, prior to analysis to determine the cellular Factor XIIa. Alternatively, cells may be obtained from a tissue sample.

If the assay used is capable of detecting or determining both cellular and non-cellular Factor XIIa, carrying out the assay on a sample comprising cells will detect or determine both the cell-bond and the non-cellular analyte. However, if the assay is carried out on a sample of isolated cells, the result will be for cellular analyte only. The term “a sample comprising cells” is used herein to denote both samples of body fluids that comprise cells and samples of isolated cells.

Cells, including cell remnants and cellular material, may be isolated, for example, as described above “Separation of forms of Factor XIIa”. For example, cells may be isolated by centrifugation and washing. Preferably the cells are centrifuged and washed at least one, preferably two or more times. Centrifugation should generally be carried out under sufficiently high g forces that the cells form a discrete pellet that can be separated from the supernatant. The pellet may be washed in a suitable medium that does not affect the cellular Factor XIIa, for example, that does not cause cellular Factor XIIa to become dissociated from cells. For example, phosphate buffered saline pH7.4 may be used for washing and for suspension of cells for the detection or detection and/or determination of cellular Factor XIIa. Flow cytometry may be used to isolate cells.

If cellular Factor XIIa has been separated from other binding forms of Factor XIIa before the assay is carried out, an assay that does not discriminate between cellular Factor XIIa and other binding forms of Factor XIIa may be used i.e. a “general” Factor XIIa assay. It may, however, be advantageous to use an assay that is capable of detecting or determining cellular Factor XIIa preferentially in relation to other forms even after a prior separation step.

If no separation step is carried out, the assay used should be capable of detecting or determining the Cellular Factor XIIa under investigation.

The presence of cellular Factor XIIa, in a tissue sample may be detected using an immunohistological technique. For example, a monoclonal antibody as described below that is labeled with an appropriate label, for example, a fluorescent label, may be used.

### Lipid Bound Factor XIIa

The present invention provides a method which comprises detecting or determining lipid bound 53 Kd Factor XIIa in a sample comprising tissue or, especially, a body fluid obtained from a mammalian subject, generally a human.

Measurement of lipid bound Factor XIIa may be performed on a sample of a body fluid, for example, whole blood or plasma. Alternatively, a lipid faction can be isolated from a body fluid or tissue and the Factor XIIa content of the lipid faction determined. A lipid fraction may be isolated as described above under “Separation of forms of Factor XIIa”. For example, lipoproteins may be isolated from a tissue or body fluid, for example, from plasma, for example by precipitation. Suitable agents for precipitating lipoproteins are known and include, for example, reagents comprising sodium chloride, manganese chloride and heparin, and phosphotungstate reagents. Various reagents and methods are described in Demacker, P. N. M. et al. Clinical Chemistry Vol. 43, No. 4, 1997, p 663-668 and in Sharma, A. et al. Clinical Chemistry, Vol. 36, No. 3, 1990, p 529-532.

A sample, for example, plasma, may be centrifuged to remove cellular components, for example, at medium to high speed, for example, at 12,000 to 16,000 g. Lipoproteins may be precipitated using a known lipoprotein precipitation agent, for example, a reagent comprising sodium chloride, manganese chloride and heparin, for example, about 500 mN sodium chloride, about 215 mM manganese dichloride and about 500 U/ml heparin, or using a phosphotungstate precipitation agent, for example, comprising about 50 mM phosphotungstate and generally magnesium chloride.

A resulting precipitate may be isolated, for example, by centrifugation. If desired, a precipitate may be resuspended in the precipitation agent and again isolated. This procedure may be repeated, if desired, for example, two or three times. Washing may be carried out between precipitation steps.

If the lipid bound Factor XIIa has been separated from other forms of Factor XIIa before the assay is carried out, an assay that does not discriminate between different forms of Factor XIIa may be used i.e. a “general” Factor XIIa assay. It may, however, be advantageous to use an assay that is capable of detecting or determining the lipid bound Factor XIIa preferentially in relation to other forms even after a prior separation step.

If no separation step is carried out, the assay used must be capable of detecting or determining the lipid bound Factor XIIa.

In the case of an immunoassay, the lipoprotein fraction may be isolated before or after the sample is contacted with an antibody. It may be advantageous to isolate the lipoprotein fraction after contact with the antibody.

Molecular Complexes and Associations of Factor XIIa with Other Molecular Species

Samples comprising molecular complexes and associations of Factor XIIa with other molecular species, generally samples of body fluids, may be prepared for an assay according to normal practice, see above.

If desired, molecular complexes comprising two or more molecules of Factor XIIa or forms of Factor XIIa in association with low or high affinity binding partners may be separated as described above under “Separation of forms of Factor XIIa” before carrying out an assay for Factor XIIa. For example, Factor αXIIa bound to low affinity binding partners, Factor βXIIa bound to low affinity binding partners, 53 Kd Factor XIIa bound to low affinity binding partners, Factor αXIIa bound to high affinity binding partners, Factor βXIIa bound to high affinity binding partners, and 53 Kd Factor XIIa bound to high affinity binding partners, may be separated.

If molecular complexes comprising two or more molecules of Factor XIIa or forms of Factor XIIa in association with low or high affinity binding partners have been separated from other forms of Factor XIIa before the assay is carried out, an assay that does not discriminate between such forms of Factor XIIa and other forms of Factor XIIa may be used i.e. a “general” Factor XIIa assay. It may, however, be advantageous to use an assay that is capable of detecting or determining such forms of Factor XIIa preferentially in relation to other forms even after a prior separation step.

An assay that is capable of detecting or determining a form or forms of Factor XIIa under investigation in preference to other forms may be used without prior separation of the form or forms of Factor XIIa under investigation.

Suitable assays, in particular, immunoassays, are described below.

### Immunoassays

An immunoassay may be used according to the present invention to detect or determine one or more forms of 53 Kd Factor XIIa in preference to other forms, said other forms of Factor XIIa preferably being non-53 Kd forms of Factor XIIa. An immunoassay may be used in relation to any sample according to the invention.

### General Immunoassay Techniques

Methods of carrying out immunoassays are well known, see for example, Tietz Textbook of Clinical Chemistry 2^{nd} Edition” Eds. Burtis, C. A. & Ashwood, E. R., Saunders (1994); Methods in Enzymology, H. Van Vunakis and J. J. Langone (Eds), 1981, 72(B); Practice and Theory of Enzyme Immunoassays, P Tijssen, Laboratory Techniques in Biochemistry and Molecular Biology, R. J. Burden and P. H. Van Knippenberg (Eds), Elsevier, 1985; Introduction to Radioimmunoassay and Related Techniques, T. Chard, ibid, 3rd Edition, 1987; and Methods in Enzymology, H. Van Vunakis and J. J. Langone (Eds) 1981, 74(C).

Immunoassay techniques, both qualitative and quantitative, include ELISA (enzyme linked immunosorbent assays), Western blotting, fluid phase precipitation assays, coated particle assays, competitive assays, sandwich assays, including forward, reverse and simultaneous sandwich assays, and solid phase radio immunoassays (SPRIA).

An antigen-antibody complex may be detected directly, for example, by the techniques described below, or by means of a labeled antibody.

### Double Antibody Sandwich Assay

An example of an ELISA format that may be used according to the present invention, is a so-called “double antibody sandwich” assay, in which an antibody, especially a monoclonal antibody, that is capable of binding to one or more forms of 53 Kd Factor XIIa, is immobilized on a solid phase support, for example, on a plastics or other polymeric material, for example on the wells of plastics microtitre plates, or on beads or particles, for example, as used in proprietary systems, for example, the IMx system of Abbott Laboratories, Abbott Park, Ill., USA. This antibody is called a “capture antibody”. A sample is incubated in contact with the immobilised capture antibody. Any form of 53 Kd Factor XIIa that is capable of binding to the immobilized antibody will be “captured” by the immobilized antibody and hence itself immobilized on the solid phase. 53 Kd Factor XIIa that is captured on the solid phase is detected using a labeled antibody that is capable of binding to one or more form of 53 Kd Factor XIIa. This labeled antibody is often called an antibody “conjugate”. By careful selection of the antibodies and/or of other assay conditions, it is possible to optimize the assay such that it preferentially measures, detects and/or determines one or more particular forms of 53 Kd Factor XIIa over other forms of Factor XIIa, said other forms of Factor XIIa preferably being non-53 Kd forms of Factor XIIa.

### Labelled Antibodies

A labelled antibody used to detection or detection and/or determination of a target antigen may be polyclonal or monoclonal. Anti-human antibodies, for example, anti-human polyclonal antibodies, are often convenient for use as labelled antibodies for clinical applications. Alternatively, an antibody that binds to the form of Factor XIIa under investigation may be used. Such an antibody may bind, for example, to the heavy chain of 53 Kd Factor XIIa.

The label may be detectable directly or indirectly. Any appropriate radioisotope may be used as a directly detectable label, for example a β-emitter or a γ-emitter, examples being ¹²⁵I, ¹³¹I, ³H, and ¹⁴C. For commercial use, non-radioactive labels, generally enzyme labels, are preferred. Enzyme labels are detectable indirectly. An enzyme label is, for example, alkaline phosphatase or a peroxidase, for example, horseradish peroxidase. An appropriate substrate for the chosen enzyme, for example, a substrate that gives rise to a detectable optical or fluorescence change, for example, phenolphthalein monophosphate or a fluorescent substrate, for example, 4-methylumbelliferyl phosphate, is used. Alternatively, there may be used an enzyme reaction that can be followed using an electrochemical method.

A labeled antibody may be used to detect an antigen-antibody complex in, for example, an ELISA, or may form a complex with an antigen, which complex may then be detected. Flow cytometry may be used for detection.

### Competitive Assays

One or more 53 Kd forms of Factor XIIa that have been labeled, for example, radiolabelled or enzyme-labelled, may be used in a competitive assay for measurement of one or more forms of 53 Kd Factor XIIa.

### Further Immunoassay Techniques

Further immunoassay methods for detecting or determining antigens utilise direct detection of a resulting antibody-antigen complex. Examples of such techniques are Surface Plasmon Resonance, Surface Acoustic Wave and Quartz Crystal Microbalance methodologies (Suzuki M, Ozawa F, Sugimoto W, Aso S. Anal Bioanal Chem 372:301-4, 2002; Pearson J E, Kane J W, Petraki-Kallioti I, Gill A, Vadgama P. J Immunol Methods; 221:87-94, 1998; Weisch W, Klein C, von Schickfus M, Hunklinger S. Anal Chem 1996 68:2000-4, 1996; Chou S F, Hsu W L, Hwang J M, Chen C Y. Clin Chem 48:913-8, 2002).

If a labelled antibody forms a complex with an antigen, the complex may be detected or determined by flow cytometry.

### Standards and Controls

Immunoassays generally use “standards” as reference points.

A standard suitable for an assay for detection or detection and/or determination of one or more forms of 53 Kd Factor XIIa may typically comprise a solution containing known amounts of one or more appropriate forms of 53 Kd Factor XIIa. Alternatively, a standard may comprise one or more appropriate forms of 53 Kd Factor XIIa bound to a supporting material such as a solid phase. Alternatively, a non-53 kd form of Factor XIIa that shows cross reactivity with 53 kd Factor XIIa may be used as a standard. Many known assays for Factor αXIIa use Factor βXIIa as a standard and therefore using Factor βXIIa as standard for 53 kd Factor XIIa assays may bring advantages due to familiarity with use of Factor βXIIa as standard.

The materials used act as standards and controls may take various forms dependent upon the assay to be used. In some assay formats, suitable material may be in aqueous solution.

A standard suitable for an assay for detection or detection and/or determination of lipid bound 53 Kd Factor XIIa typically comprises a solution containing known amounts of lipid bound 53 Kd Factor XIIa. Alternatively, a standard may comprise 53 Kd Factor XIIa bound to a non-lipid supporting material, for example, a solid phase, or an aqueous solution of 53 Kd Factor XIIa may be used as a standard.

A standard suitable for an assay for detection or detection and/or determination of urinary 53 Kd Factor XIIa would typically comprise of a solution containing a known amount of 53 Kd Factor XIIa.

### Immunohistology

The presence of a form or forms of 53 Kd Factor XIIa in a tissue sample may be detected using an immunohistological technique. For example, a monoclonal antibody as described above, labeled with an appropriate label, for example, a fluorescent label, may be used. Typically a labeled antibody is contacted and incubated with a tissue sample, the reagents are subsequently washed off under conditions that do not disrupt any antibody-antigen complexes that have formed, and any such complexes are detected.

### Chromogenic Assays

Detection or determination of one or more 53 Kd forms of Factor XIIa may be performed by measuring its enzyme activity using a chromogenic substrate for example, as described by Vinazzer H., Thromb Res., 14, 155-66, 1979.

This assay may involve a step where one or more forms of 53 Kd Factor XIIa are isolated from other forms, see above.

### Immunoassay for Cellular 53 Kd Factor XIIa

Cells may be isolated from a body fluid, for example, from blood or plasma, for example, by centrifugation and washing, preferably at least once and especially two or more times, for example, in a suitable medium that does not affect the cellular 53 Kd Factor XIIa, for example, that does not cause cellular 53 Kd Factor XIIa to become dissociated from cells. Suitable liquids are generally buffers, for example, phosphate buffered saline (PBS), for example, at pH 7.4.

A sample of a body fluid comprising cells may be washed, centrifuged at high speed, and then suspended in a suitable liquid give “washed cells”. An example of high speed centrifugation is 16,000 g for 10 minutes. An example of a suitable washing and suspending liquid is PBS pH 7.4. One or more, for example, two or three, or more, rounds of centrifugation may be carried out.

Cell rich plasma may be obtained, for example, by low speed centrifugation of blood, for example, by centrifuging citrated blood from 10 minutes at 1000 g. Further centrifugation, for example, high speed centrifugation, of cell rich plasma, for example, centrifugation at 16,000 g for 10 minutes, gives a supernatant, called cell poor plasma.

### Immunoassay for Lipid Bound 53 Kd Factor XIIa

An immunoassay may be carried out using mAb 2/215 or an analogue thereof or a fragment thereof, for example, a Fab fragment. In the case of a capture assay, it is preferably to use mAb 2/215 or an analogue thereof as the capture antibody. Alternatively an 53 Kd Factor XIIa-specific antibody according to the invention may be use. A different antibody, for example, a different polyclonal antibody or a different monoclonal antibody, or the same antibody may be used for detection.

A direct immunoassay, for example, a radioimmunoassay, may be used. In such a case it is preferable to use a monoclonal antibody of the invention or an analogue thereof or a fragment thereof, for example, a Fab fragment. Examples of suitable labels are given above.

The lipoprotein fraction may be isolated before or after the sample is contacted with an antibody. It may be advantageous to isolate the lipoprotein fraction after contact with the antibody. The lipoprotein fraction may be isolated as described above in the “Sample preparation” section.

As an alternative to an immunoassay, detection and/or determination of lipid bound 53 Kd Factor XIIa may be performed by measuring its enzyme activity using a chromogenic substrate for example, as described by Vinazzer H., Thromb Res., 14, 155-66, 1979. This may involve a stage where one or more species are isolated from other species, for example, as described above.

Immunoassay for Molecular Complexes and Associations of 53 Kd Factor XIIa with Other Molecular Species

An immunoassay may be carried after separation of molecular complexes and associations of 53 Kd Factor XIIa with other molecular species form other forms of Factor XIIa, or on a sample without such separation. For example, if desired, molecular complexes comprising two or more molecules of 53 Kd Factor XIIa or forms of 53 Kd Factor XIIa in association with low or high affinity binding partners may be separated as described above under “Separation of forms of Factor XIIa” before carrying out an assay for 53 Kd Factor XIIa. For example, Factor αXIIa bound to low affinity binding partners, Factor βXIIa bound to low affinity binding partners, 53 Kd Factor XIIa bound to low affinity binding partners, Factor αXIIa bound to high affinity binding partners, Factor βXIIa bound to high affinity binding partners, and 53 Kd Factor XIIa bound to high affinity binding partners, may be separated.

Any of the immunoassays described above may be used to determine molecular complexes and associations of Factor XIIa with other molecular species. As described above, it may be preferable to use mAb 2/215 or an analogue thereof as an antibody or a 53 Kd Factor XIIa-specific antibody according to the invention, in particular as the capture antibody in a capture immunoassay. The labelled antibody used for detection should be capable of binding to the captured form of Factor XIIa. For example, the labelled antibody may bind to the heavy chain of Factor αXIIa, to Factor βXIIa, or to 53 Kd Factor XIIa.

### Immunoassay and Other Assays for Urinary 53 Kd Factor XIIa

Any of the immunoassays described above may be used to determine one or more forms of 53 Kd Factor XIIa in urine preferentially relative to other molecular weight forms. As described above, it may be preferable to use mAb 2/215 or an analogue thereof is used as an antibody or a 53 Kd Factor XIIa-specific antibody according to the invention, in particular as the capture antibody in a capture immunoassay.

### Kits

The present invention further provides a kit for

carrying out an immunoassay of the present invention,
which kit comprises, each in a separate container or
otherwise compartmentalised: (i) a monoclonal antibody that is capable of binding to one or more forms of 53 Kd Factor XIIa, and (ii) a labeled antibody capable of binding to one or more forms of 53 Kd Factor XIIa when one or more forms of 53 kd Factor XIIa is bound to the monoclonal antibody defined in (i) both antibodies characterized in that they have a corrected cross reactivity with one or both of Factor αXIIa and Factor βXIIa of 10% or less, more preferably 5% or less, still more preferably 2% or less, still more preferably 1% or less, still more preferably 0.5% or less, still more preferably 0.1% or less.

The kit may comprise further components for carrying out an immunoassay, for example, as described above. The monoclonal antibody may be immobilised on a solid support.

A kit according to the invention may comprise, for

example,
a) a monoclonal antibody that is capable of binding to one or more forms of 53 Kd Factor XIIa,
(b) a standard typically comprising of a solution containing known amounts of one or more forms of Factor XIIa
(c) labelled antibody capable of reacting with one or more forms of 53 Kd Factor XIIa when one or more forms of 53 Kd Factor XIIa is bound to the monoclonal antibody defined in (i).

The materials used act as standards and controls may take various forms dependent upon the assay to be used. In some assay formats, suitable material may be in aqueous solution. In other formats, for example where the same antibody is used as the capture and detection (conjugate) antibody in an ELISA, it may be desirable to create constructs containing multiple Factor XII molecules or fragments thereof, including the various forms of Factor XIIa, for example, by binding 53 Kd Factor XIIa to the surface of beads, for example, polycarbonate beads, for example, 3 μM in diameter.

Further examples of standards are given above.

Alternatively, a kit may comprise labeled forms of Factor XIIa, especially a labeled form of 53 Kd Factor XIIa, for use in a competitive assay.

A kit may also comprise further components, each in

a separate container, for example, diluent(s), wash reagent solution(s) and substrate solution(s).

### Assay Devices

The present invention also provides an assay device suitable for carrying out an assay of the invention. The term “assay device” is used herein to denote means for carrying out an immunoassay comprising a solid phase, generally a laminar solid phase, for example, a membrane, sheet, strip, coating, film or other laminar means, on which is immobilized an appropriate capture antibody. The immobilized antibody is preferably present in a defined zone, called herein the “antigen capture zone”.

An assay device may incorporate the solid phase within a rigid support or a housing, which may also comprise some or all of the reagents required for carrying out an assay. Sample is generally applied to an assay device at a predetermined sample application zone, for example, by pouring or dripping the sample on the zone, or by dipping the relevant part of the device into the sample. If the sample application zone is at a different site from the antibody capture zone, the arrangement of the device is generally such that antigens in the sample migrate to the antibody capture zone. The required reagents are then applied in the appropriate order at designated application zones, which may or may not be the same as the sample application zone. Again, if the or any reagent application zone is at a different site from the antibody capture zone, the arrangement of a device is generally such that the reagent(s) migrate to the antibody capture zone, where any antigen-antibody complex formed is detected. All or some of the reagents required for an immunoassay may be incorporated within a device, in liquid or dry form. If so, a device is generally arranged such that interactions between different parts of the device, which interactions may occur automatically during the operation of the device or may be brought about by the user of the device, bring the various reagents into contact with one another in the correct sequence for the immunoassay to be carried out.

A wide variety of assay devices are described in the literature of immunoassays. Examples of membrane devices are described in U.S. Pat. Nos. 4,623,461 and 4,693,984. Depending on their design and their speed of action, some assay devices are called “dipsticks” and some are called “rapid assay” devices. A “rapid assay” device generally provides a result within ten minutes of the application of sample. (A typical microtitre plate or bead assay requires incubation steps, and generally takes at least an hour to provide a result.) Accordingly, although assay devices are generally more expensive than microtitre or bead format assays, they have particular uses in clinical testing, for example, when a result is required rapidly, for example, in the case of emergency treatment.

Assay devices have the particular advantage that they can be used without the need for sophisticated laboratory facilities or even without the need for any laboratory facilities. They may therefore be used for “Point of Care” testing, for example, in an emergency room, in a doctor's surgery, in a pharmacy or, in certain cases, for home testing. They are particularly useful in territories where laboratory facilities are few and far between.

### Antibody Cross Reactivity

The antibodies of the invention have corrected cross-reactivity with one or both of Factor αXIIa and Factor βXIIa of 10% or less, more preferably 5% or less, still more preferably 2% or less, still more preferably 1% or less, still more preferably 0.5% or less, still more preferably 0.1% or less. Preferably, the antibodies have a low cross reactivity, for example of 0.5% or less or more preferably of 0.1% or less with Factor XII. A factor to take into consideration in assessing the cross-reactivity of an antibody of the invention with Factor αXIIa, Factor βXIIa and Factor XII is that even “pure” preparations of such proteins are almost inevitably contaminated with small amounts of 53 Kd Factor XIIa. Likewise as explained in Silverberg and Kaplan, Blood 60, 1982, 64-70 preparations of Factor XII are inevitably contaminated with Factor XIIa. WO90/08835 gives details of methods of assessing the corrected cross-reactivity with Factor XII such methods are applicable to the assessing the corrected cross reactivity with Factor αXIIa and Factor βXIIa. Unless specified otherwise, the term “cross reactivity” is used herein to mean the corrected cross reactivity.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495).

WO90/08835, which is incorporated herein by reference, describes in general terms how to produce an antibody that binds to Factor αXIIa and to Factor βXIIa and that has shows a corrected cross-reactivity with Factor XII of 0.1% or less, and gives specific details of the production of mAb 2/215 and mAb 201/9. The general and specific methods described therein may used to produce a monoclonal antibody suitable for use according to the present invention, for example, a monoclonal antibody binding to 53 Kd Factor XIIa but not binding to one or more non-53 Kd forms of factor XIIa.

A general protocol for producing monoclonal antibodies suitable for use according to the present invention, based on the disclosure of WO90/08835, is given in Example 22 of WO04/057343 which is incorporated herein by reference.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495). The antigen used in the production of monoclonal antibodies may be Factor αXIIa or 53 Kd factor XIIa. Resulting monoclonal antibodies may be screened for those that show no significant binding to one or more non-53 Kd form of Factor XIIa, for example, having a corrected cross-reactivity with Factor αXII or βXII of 0.1% or less.

Resulting monoclonal antibodies may be screened for binding to the form of 53 Kd Factor XIIa to which binding is desired, for example, cellular 53 Kd Factor XIIa, lipid bound 53 Kd Factor XIIa or a complex or association of 53 Kd Factor XIIa with other Factor XIIa molecules or with high or low binding affinity partners.

It may be advantageous to use monoclonal antibody 2/215 or 201/9, respectively, as a reference antibody in screening for antibodies that bind to specific binding forms of 53 Kd Factor XIIa. A selected antibody may have binding characteristics for selected forms of 53 Kd Factor XIIa that are the same as or similar to or different to those of mAb 2/215 or 201/9, respectively.

The invention is not limited to hybridomas of murine or part-murine origin. Both fusion partners (spleen cells and myelomas) may be obtained from any suitable animal. Recombinant antibodies may be produced. Antibodies may be brought into chimeric or humanized form, if desired. Hybridomas are preferably cultured in vitro.

### Polyclonal Antibodies

The present invention also provides polyclonal antibodies, also called a polyclonal antiserum, that are capable of reacting selectively with one or more forms of 53 Kd Factor XIIa. Such antibodies may be labeled and used for detection of one or more captured forms of 53 Kd Factor XIIa, in an ELISA.

The invention also provides a method for the production of such a polyclonal antiserum, which comprises administering an antigen present in the 53 Kd form of Factor XIIa to an animal, obtaining serum from the animal, screening the serum for binding to one or more forms of 53 Kd Factor XIIa. In some cases, Factor XII or a non-53 Kd form of Factor XIIa can be used as the antigen.

### Urine Testing

The invention also includes a method which comprises detecting or determining 53 Kd Factor XIIa in a sample comprising urine obtained from a subject. In this embodiment of the invention it may not be necessary to detect or determine any one of more binding forms of 53 Kd Factor XIIa preferentially in relation to other binding forms of Factor XIIa. An assay that does not discriminate between binding forms may be used. Such an assay may be, for example, a chromomeric assay or an immunoassay.

Assay of 53 Kd Factor XIIa in urine by means of an assay that can discriminate between different molecular weight forms of Factor XIIa, provides useful information in relation to renal function, renal disease and renal damage, because Factor XIIa concentrations in urine are a sensitive marker of renal function, renal disease and renal damage, particular in conditions where extensive proteinuria is not present. Elevated concentrations of Factor XIIa in urine of a subject, for example, relative to healthy subjects, are indicative of any one of impaired renal function, renal disease and renal damage. Changes in the concentration of urinary Factor XIIa may be indicative of change in a clinical condition, for example, exacerbation of the condition or improvement, for example, in response to therapy.

### Clinical and Other Utility

The invention, especially the immunoassays described above, provides a method of detection and/or determination of different forms of 53 Kd Factor XIIa that can be used readily on automated equipment for large scale use.

Factor XII and its predominant activated form, 53 Kd Factor XIIa, are considered to be involved in blood coagulation and other contact systems, also known as contact phase systems, for example, fibrinolysis, complement cascade, inflammation and vaso-dilation, see Jacobsen S. and Kriz M., Br J. Pharmacol., 29, 25-36, 1967; Kurachi K et al, Biochemistry, 19, 1330-8 1980; Radcliffe R et al, Blood, 50, 611-7, 1977; Ghebrehiwet B et al, J Clin Invest, 71, 1450-6. 1983; Z Toossi et al, Proc Natl Acad Sci USA, 89, 11969-72, 1992; Wachtfogel Y T et al, Blood 67, 1731-7, 1986; Wachtfogel Y T et al, Thromb Haemost, 80, 686-91, 1998; and Schreiber et al AD, J Clin Invest., 52, 1402-9, 1973.

As Factor XII and its predominant activated form, 53 Kd Factor XIIa, are involved in haemocoagulation and have a role in maintaining vascular wholeness and blood pressure, in influencing various functions of endothelial cells, in control of fibrinolysis and in maintaining the constitutive anticoagulant character of the intravascular space, measurement of specific forms of 53 Kd Factor XIIa is useful in investigations of those systems, including for example, fibrinolysis, complement cascade, and vasodilation, and also in investigations relating to thrombosis and stenosis.

Clinical and experimental studies indicate that the contact system, which includes 53 Kd Factor XIIa, is involved in acute and chronic inflammation, shock of different aetiologies including septic shock, diabetes, allergy, thrombo-haemorrhagic disorders including disseminated intravascular blood coagulation, oncological diseases, cardiovascular conditions, for example, myocardial infarction, angina and acute coronary syndrome, angiogenesis, sepsis, spontaneous abortion and thromboembolism.

The involvement of 53 Kd Factor XIIa in haemocoagulation, in maintaining vascular wholeness and blood pressure, in control of fibrinolysis and in maintaining the constitutive anticoagulant character of the intravascular space supports the clinical and experimental observations of the involvement of 53 Kd Factor XIIa in thrombo-haemorrhagic disorders including disseminated intravascular blood coagulation, oncological diseases, cardiovascular conditions, for example, myocardial infarction, angina and acute coronary syndrome, angiogenesis, and thromboembolism.

Factor XIIa, including Factor XIIa in its predominant 53 Kd form, is present on granulocytes, which are activated/involved in the inflammatory process. This observation supports the clinical and experimental studies that implicate Factor XIIa in various conditions that involve inflammation, for example, acute and chronic inflammation, shock of different aetiologies including septic shock, allergy, oncological diseases, and sepsis.

Detection and/or determination of specific forms of 53 Kd Factor XIIa, are therefore likely to be useful in clinical and scientific investigations of diseases and disorders in which the contact system may be involved, including diagnosing, monitoring, or predicting the susceptibility to, progress of, or outcome of such a disease or disorder, or of treatment of the disease or disorder in a subject having or suspected of having the disease or disorder. Such diseases and disorders include acute and chronic inflammation, shock of different aetiologies, diabetes, allergy, thrombo-haemorrhagic disorders including disseminated intravascular blood coagulation and thromboembolism, thrombosis and stenosis, oncological diseases, cardiovascular conditions, for example, myocardial infarction, angina, acute coronary syndrome, angiogenesis, sepsis, and spontaneous abortion.

Detection or determination of one or more forms of 53 Kd Factor XIIa, is therefore useful as an aid to diagnosing, monitoring, or predicting the susceptibility to, progress of, or outcome of a disease or disorder, or of treatment of the disease or disorder in a subject having or suspected of having the disease or disorder, in which disease or disorder the amount of one or more 53 Kd forms of Factor XIIa is different from that in healthy subjects. Changes in the concentration of one or more 53 Kd forms of Factor XIIa may be indicative of any of the diseases and disorders mentioned above. Changes in concentration in a subject with time may be indicative of change in the condition, for example, exacerbation of the condition, or improvement, for example, in response to therapy. Such methods of diagnosing, monitoring, predicting the susceptibility to, progress of, or outcome of a disease or disorder, or of treatment of the disease or disorder, called “diagnosis, prediction and monitoring”, are part of the present invention.

In addition, Factor XIIa in urine is known to be a sensitive marker of renal function, renal disease and renal damage, and it is therefore likely that detection or determination of 53 Kd Factor XIIa in urine can provide useful information on renal function, renal disease and renal damage.

### Diagnosis, Prediction and Monitoring

The present invention provides a method for diagnosing, monitoring, or predicting the susceptibility to, progress of, or outcome of a disease or disorder, or of treatment of the disease or disorder in a subject having or suspected of having the disease or disorder, which comprises detecting or determining one or more forms of 53 Kd Factor XIIa in preference to other forms of Factor XIIa, said other forms of Factor XIIa preferably being non-53 Kd forms of Factor XIIa, in a sample obtained from the subject, and comparing the results obtained for the subject with the results obtained using the same assay for samples obtained from at least any one or more of the following:

(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

The sample may be any of those described above. For example, the sample may be a sample of a body fluid, for example, blood, plasma, serum, urine, cerebrospinal fluid, saliva, or tears.

The assay may be for the detection and/or detection and/or determination of one or more 53 Kd forms of Factor XIIa, for example, any one or more selected forms, for example, any one or more of cellular 53 Kd Factor XIIa, lipid bound 53 Kd Factor XIIa and urinary Factor 53 Kd XIIa.

Specificity of an assay for one or more forms of 53 Kd Factor XII over other, preferably non-53 Kd, forms of Factor XIIa may be achieved or improved by design of the assay, as described above. In the case of an immunoassay, such design may include any one or more of choice of the antibody or combination of antibodies to be used; presence, absence and choice of a detergent; and conditions used for plate coating in the case of an antigen capture assay involving an antibody coated on a solid phase, see above.

The assay for 53 Kd Factor XIIa may be an immunoassay that comprises the use of an antibody that is capable of binding to the form or forms of 53 Kd Factor XIIa under investigation. In such an assay an antibody that is capable of binding to the form or forms of 53 Kd Factor XIIa under investigation is immobilized on a solid phase as a capture antibody.

Alternatively or in addition, an antibody that is capable of binding to the form or forms of 53 Kd Factor XIIa under investigation is labeled with a label that is detectable directly or indirectly.

In an immunoassay an antibody that is capable of binding to the form or forms of 53 Kd Factor XIIa under investigation may be mAb 2/215 or an analogue thereof, mAb 201/9 or an analogue thereof, or a polyclonal antibody that is capable of binding to 53 Kd Factor XIIa. However, if an antibody such as mAb 2/215 is used which is unable to distinguish between 53 Kd Factor XIIa and non-53 Kd form of Factor XIIa is used, it will be necessary to use the assay in conjunction with a method of separating or distinguishing different molecular weight forms of Factor XIIa.

In an immunoassay in which mAb 2/215 or an analogue thereof, mAb 201/9 or an analogue thereof, or a polyclonal antibody that is capable of binding to 53 Kd Factor XIIa is used, the antibody may be labeled with a label that is detectable directly or indirectly and/or may be immobilized on a solid phase as a capture antibody.

53 Kd Factor XIIa captured by the defined antibody may be detected or determined using a labeled antibody, for example, as defined above.

The disease or disorder under investigation may be any of those described above in the “Clinical utility” section, for example, diseases and disorders of the coagulation system; conditions that involve hemaocoagulation, fibrinolysis, kininogensis, complement activation or angiogenesis, maintaining vascular wholeness and blood pressure, maintaining the constitutive anticoagulant character of the intravascular space, or tissue defence and repair; conditions that involve acute or chronic inflammation, shock of any aetiology, diabetes, allergy, a thrombo-haemorrhagic disorder, sepsis, spontaneous abortion or an oncological disease; and conditions that involve intravascular blood coagulation or thromboembolism, thrombosis or stenosis, myocardial infarction, acute coronary syndrome or angina.

Treatment of the clinical or pathological condition may involve administration of a therapeutic agent and/or may involve a surgical procedure. For example, treatment of thrombosis or stenosis may involve coronary artery angioplasty and/or thrombolysis.

It may be advantageous to test a series of samples obtained from a subject, for example, samples obtained during the course the disease or disorder and/or samples obtained during treatment of the disease or disorder and/or before treatment is started.

The disease or disorder may be or involve thrombosis or stenosis and/or treatment may involve coronary artery angioplasty or thrombolysis.

As stated above, Factor XIIa in urine is a sensitive marker of renal function, renal disease and renal damage.

The present invention relates to a method for diagnosing or monitoring diseases or disorders in which 53 Kd Factor XIIa, in particular the concentration of 53 Kd Factor XIIa in the urine of a subject having the disease or disorder is different from that in a healthy subject.

The present invention provides a method for diagnosing or monitoring a disease or disorder, or monitoring treatment of the disease or disorder, which comprises detecting or determining 53 Kd Factor XIIa, in particular the concentration of 53 Kd Factor XIIa, in the urine of a subject having or suspected of having the disease or disorder.

For example, the present invention provides a method for diagnosing or monitoring renal function, renal disease or renal damage, or monitoring treatment of impaired renal function, renal disease or renal damage in a subject having or suspected of having impaired renal function, renal disease or renal damage, which comprises detecting or determining 53 Kd Factor XIIa in a sample obtained from the subject.

Generally the results obtained for the subject are compared with the results obtained using the same assay for samples obtained from at least any one or more of the following:

(i) subjects having the disease or disorder, for example, impaired renal function, renal disease or renal damage;
(ii) subjects having the disease or disorder, for example impaired renal function, renal disease or renal damage, which subjects were monitored in relation to the progress and/or outcome of the disease or disorder, for example impaired renal function, renal disease or renal damage;
(iii) subjects having the disease or disorder, for example impaired renal function, renal disease or renal damage and having the treatment therefor;
(iv) subjects having the disease or disorder, for example impaired renal function, renal disease or renal damage and the treatment, which subjects were monitored in relation to the treatment in relation to the progress and/or outcome of the disease or disorder, for example impaired renal function, renal disease or renal damage;
(v) subjects who do not have the disease or disorder, for example impaired renal function, renal disease or renal damage;
(vi) the same subject before the onset of the disease or disorder, for example impaired renal function, renal disease or renal damage or before the start of the treatment of the disease or disorder, for example impaired renal function, renal disease or renal damage; and
(vii) the same subject at an earlier or later stage of the disease or disorder, for example impaired renal function, renal disease or renal damage or the treatment, or before the onset of the disease or disorder, for example impaired renal function, renal disease or renal damage.

The 53 Kd Factor XIIa may be detected or determined by an assay that is capable of detecting or determining one or more form of 53 Kd Factor XIIa preferentially relative to other forms of Factor XIIa, said other forms of Factor XIIa preferably being non-53 Kd forms of Factor XIIa.

The following non-limiting Examples illustrate the present invention.

### EXAMPLES

### Example 1

In this example the existence of the 53 Kd species of activated Factor XII in plasma was demonstrated by binding to antibody fragments labelled with a radiotracer (iodine 125), and separating the resultant complexes on the basis of molecular weight using high performance liquid chromatography (HPLC).

Fab antibody fragments of antibody 2/215 were prepared using an “Immunopure Fab Preparation Kit” (Pierce, 3747 N Meridian Road, PO Box 117, Rockford, Ill. 61105, U.S.A.) according to the manufacturer's instructions. The Fab fragments were then radiolabelled with iodine 125 by Amersham Pharmacia Biotech (Pollards Wood, Nightingales Lane, Chalfont St Giles, HP8 4SP United Kingdom).

1 μl of radiolabelled antibody was added to 1 ml of plasma from each of a number of healthy human volunteers. After incubation for 4 hours, the components of the plasma were separated by High Performance Liquid Chromatography (HPLC). The HPLC system used was an Agilent 1100 system obtained from Agilent Technologies, Santa Clara, Calif., USA.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min⁻¹ and the injection volume was 100 μl.

The HPLC eluant was monitored by measuring the absorbance at 280 nm, and by monitoring radioactivity using a Flow-Count Radiochromatography detector (LabLogic, Sheffield, UK).

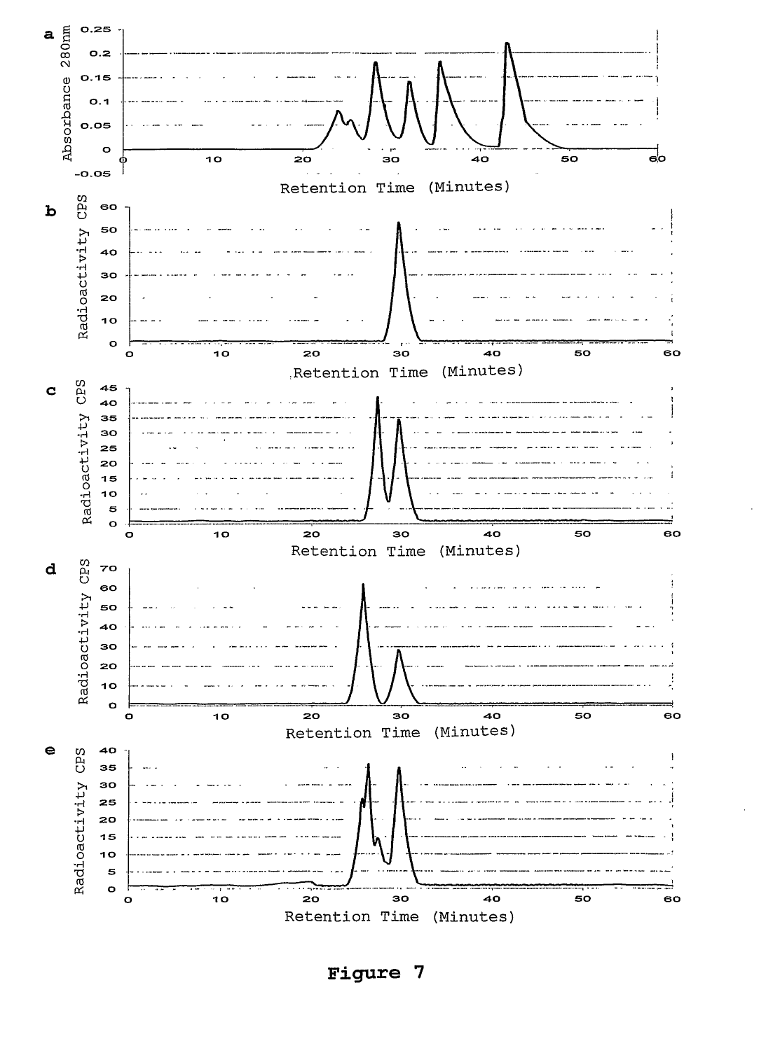

An example of a plot of radioactivity versus time is shown in FIG. 7, where it can be seen that the largest peak due to association of the antibody fragment with a species in the plasma has a molecular weight of around 83 Kd, indicating association of the 30 Kd radiolabelled Fab with a plasma species of around 53 Kd.

### Example 2

Mass spectroscopy was performed using a Ciphergen Surface Enhanced Laser Desorption and Ionisation-Time of Flight (SELDI-TOF) system (Ciphergen Biosystems, Inc., Fremont, Calif., USA).

Monoclonal antibodies 2/215 and 201/9, which were raised against activated Factor XII, and a non-specific murine monoclonal antibody acting as a control, were coupled to pre-activated RS-100 SELDI-TOF chips (Ciphergen) by the addition of 2 μL of the antibody solution (0.2 mg/ml in PBS) and 3 μL of 50 mM NaHCO3 (pH 8.2) to each spot of the arrays and incubated for 1 hour at room temperature in a humid chamber. Following incubation, the antibody solution was removed and 4 μL of blocking solution (2 mg/ml bovine serum albumin in PBS) was added to each spot and incubated for 20 minutes at room temperature in a humid chamber. Following removal of the blocking solution, each array was washed twice in 15 ml PBS for 5 minutes.

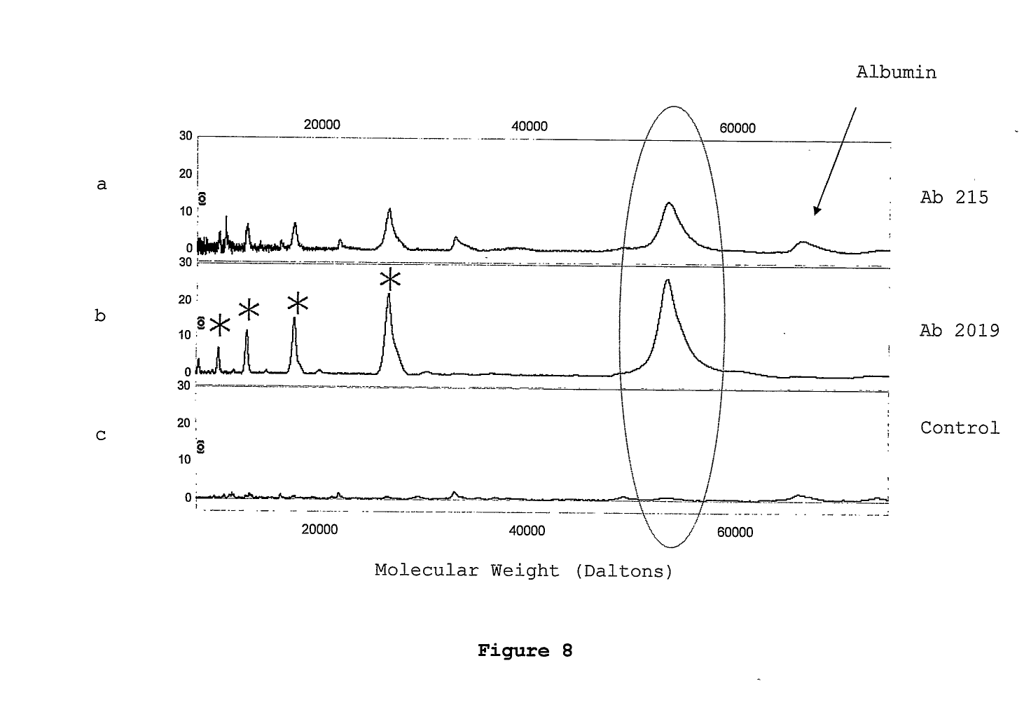

100 μl plasma and 200 μl PBS were applied to each spot on the ProteinChip Array using a 96 well bioprocessor (Ciphergen). The samples were then incubated at room temperature for 50 minutes on a platform shaker. The array then underwent three washes of 5 minutes each with PBS with 0.05% Triton X-100 (200 ul per spot). Each spot was then washed twice more for 5 minutes with 200 μl PBS per spot. Each array was then rinsed in 15 ml distilled water for 5 sec. After air drying 0.5 ml of saturated EAM1 (Ciphergen) in 500 ml/L acetonitrile, 5 ml/L trifluoroacetic acid was applied twice to each spot. Proteins bound to the antibody immobilised the RS100 ProteinChip arrays were detected using the ProteinChip Reader. FIG. 8 shows that the antibodies raised against activated Factor XII (2/215 and 201/9, labeled respectively as “Ab 215” and “Ab 2019”) react with a plasma component of around 53 Kd, whilst this species is not observed interacting with the control antibody (FIG. 8).

### Example 3

80 ul Interaction Discovery Beads (Ciphergen) were washed three times with 500 μL sodium acetate buffer pH 5. The beads were then distributed equally between 4 Eppendorf tubes and 40 μg of the antibody in 1.5 mL of 50 mM sodium acetate buffer pH 5.0 was added to each tube, and incubated overnight at 4° C. on a shaker. Following removal of the supernatant (antibody solution) the beads were washed once with 1000 μL of 50 mM sodium acetate buffer pH 5.0. The beads were then incubated with 1000 μL of blocking solution (2 mg/mL BSA in PBS) for 20 min on a vortex mixer at room temperature, and washed twice with 1000 μL PBS, 0.02% Triton X-100 and once with 500 μL 1×PBS.

300 μl plasma plus 600 μl PBS, 0.02% Triton X-100 was added to the beads and Incubated for 1 hour on a vortex mixer at room temperature. The beads were then washed twice for 15 minutes with 1000 μL PBS, 0.02% Triton X-100 and twice for 15 minutes with 1000 μl PBS and once for 15 seconds with 1000 μL water. 40 μl sample buffer was added to elute the proteins.

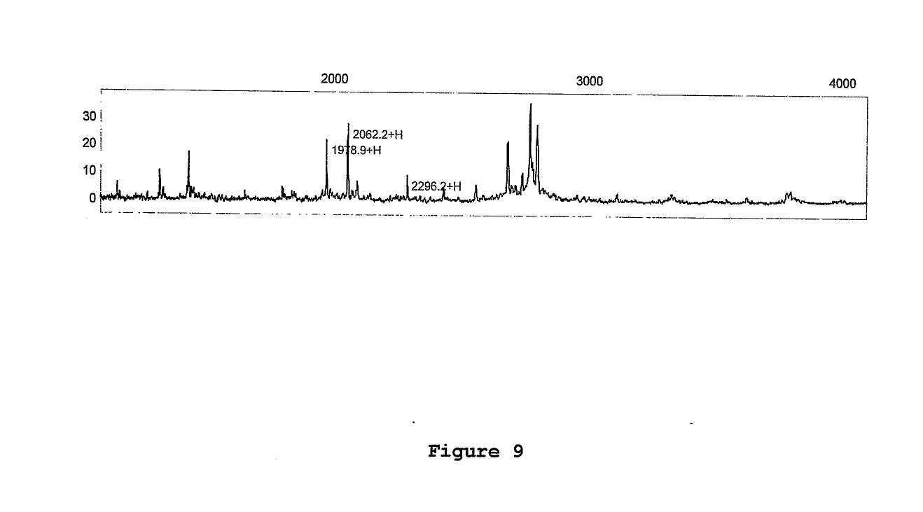

The eluate was the submitted to gel electrophoresis, and the band running at around 53 Kd was excised and submitted to tryptic digest. Fenselau, C. 1997. MALDI-MS and strategies for protein analysis. Anal. Chem. 661A-665A. Jungblut, P. and Thiede, B. 1997. Protein identification from 2-DE gels by MALDI mass spectrometry. Mass Spectrom. Rev. 16:145-162. Patterson, S. D. and Aebersold, R. 1995. Mass spectrometric approaches for the identification of gel-separated proteins. Electrophoresis 16:1791-1814. The digest was then submitted to MALDI-TOF (matrix assisted laser desorption ionisation-Time of Flight mass spectrometry) analysis using a Ciphergen ProteinChip Reader. A portion of the resultant peptide pattern is shown in FIG. 9. Comparison of the resultant peptide pattern with peptide patterns in databases of known proteins indicated that the 53 Kd protein was derived from Factor XII but missing approximately 115 amino-terminal residues.

### Example 4

This example demonstrates that measurement of the 53 Kd form of Factor XIIa provides a prediction of risk of all cause mortality in patients admitted to hospital with suspected myocardial infarction and/or acute coronary syndrome.

Data was obtained on 871 patients admitted to the hospital. Each patient had Factor XIIa measured using assays preferentially measuring the 53 kD form of Factor XIIa. Data from these assays were studied to ascertain if it provided prediction of the primary clinical endpoint of all cause mortality.

The prognostic utility of the assays was determined by ranking the 53 kD Factor XIIa values (from lowest to highest) and then splitting the population into quartiles i.e. the 25% of individuals with the lowest 53 kD Factor XIIa concentrations were in the 1st quartile, whilst the 25% of individuals with the highest concentrations were in the 4th quartile.

The 53 Kd form of XIIa was measured using high performance liquid chromatography following reaction of the sample with Iodine-125 labelled antibody.

Fab antibody fragments of antibody 2/215 were prepared using an “Immunopure Fab Preparation Kit” (Pierce, 3747 N Meridian Road, PO Box 117, Rockford, Ill. 61105, U.S.A.) according to manufacturer's instructions. These Fab fragments were then radiolabelled with Iodine-125 by Amersham Pharmacia Biotech (Pollards Wood, Nightingales Lane, Chalfont St Giles, HP8 4SP United Kingdom).

1 μl of radiolabelled antibody was added to 1 ml of plasma from each of a number of healthy volunteers. After incubation for 4 hours, the components of the plasma were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min-1 and the injection volume was 100 μl.

The HPLC eluant was monitored by measuring the absorbance at 280 nm, and by monitoring radioactivity using a Flow-Count Radiochromatography detector (LabLogic, Sheffield, UK)

Molecular weight standards were run, and from comparison with these the 53 kD XIIa peak could be identified. Integration of the area under this peak (radioactivity signal) provided a quantitative measure of the 53 kD form of XIIa. Calibration of quantitation was obtained by running standards with known quantities of the 30 kD form of XIIa (βXIIa).

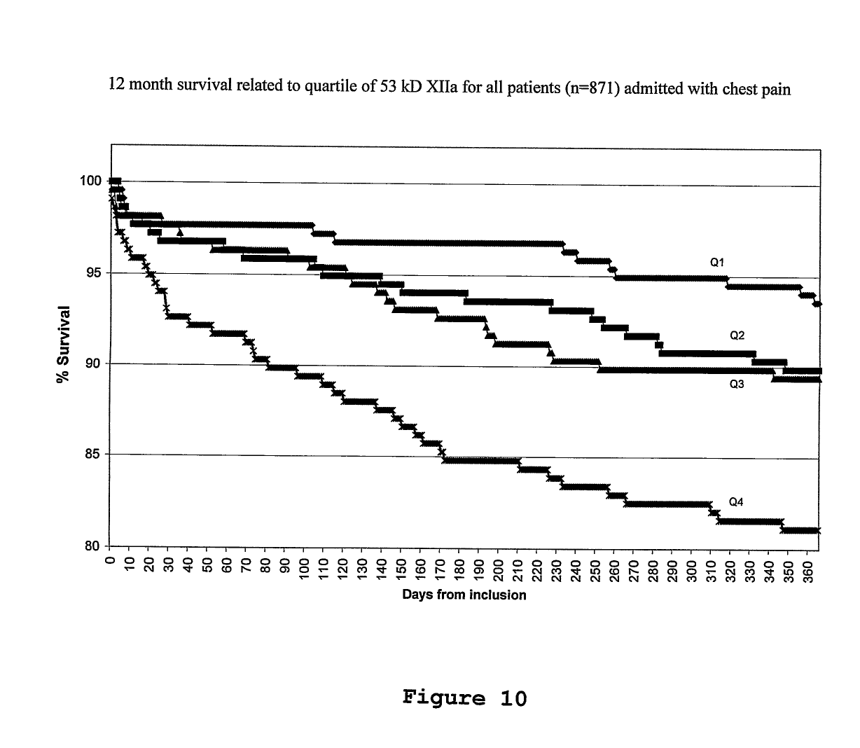

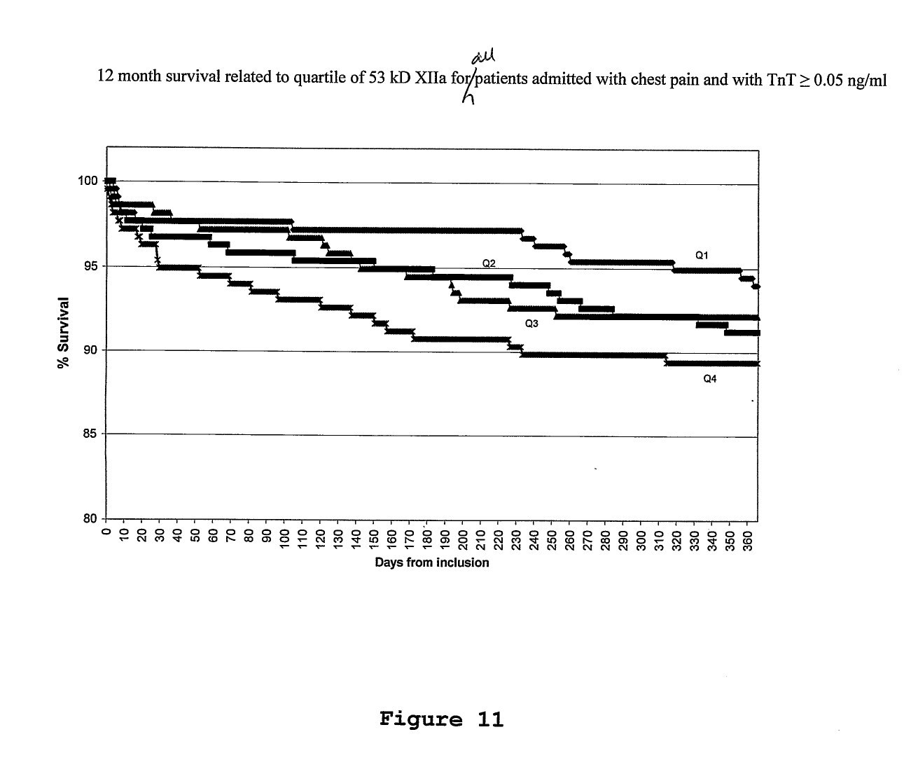

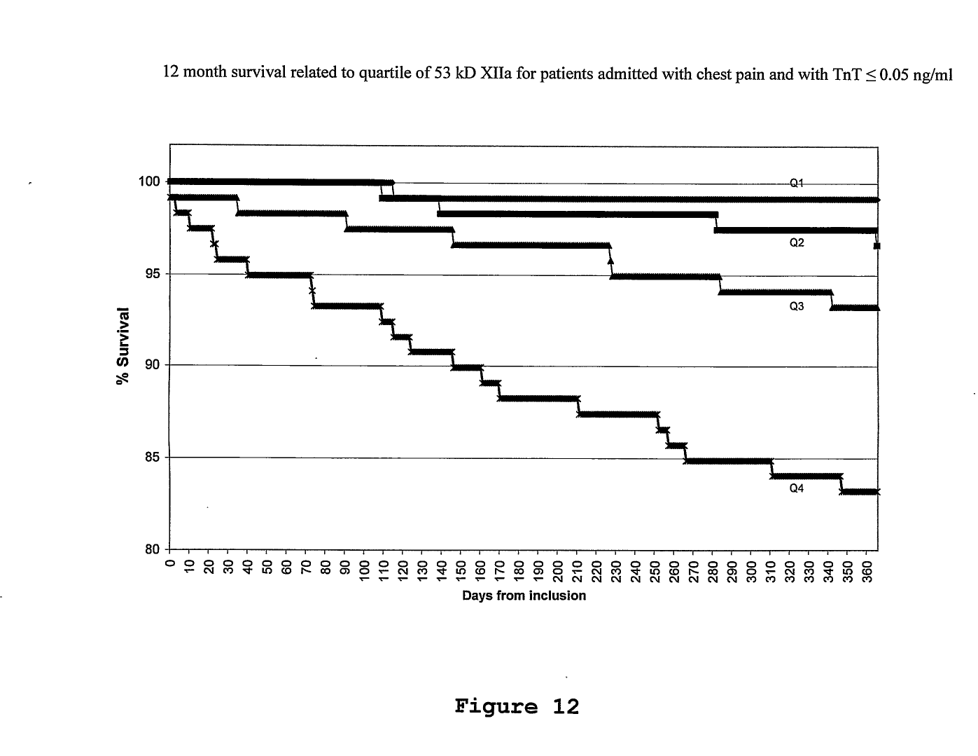

Table I shows the relative risk of all cause mortality related to the concentration of the 53 kD form of XIIa at different follow-up timepoints. In all cases those patients with the highest 53 kD XIIa concentration were at statistically significant increased risk of death. This was true for all patients, patients admitted with myocardial infarction (defined as admission Troponin T (TnT) greater than 0.05 mg/ml but particularly in patients admitted with Troponin negative (TnT less than or equal to 0.05 ng/ml) chest pain. FIGS. 10 to 12 show Kaplan Meier survival plots for all patients, patients who had admission TnT greater than 0.05 ng/ml and patients who had admission TnT less than or equal to 0.05 ng/ml respectively.

TABLE I
Odds ratios for all cause mortality pertaining to 53 kD XIIa concentration.
53 kD Factor XIIa quartile

| 53 kD Factor XIIa quartile | | Q1 | Q2 | Q3 | Q4 |
|---|---|---|---|---|---|
| (Range pM) | | (<25.0) | (25.0-35.0) | (35.1-55.0) | (>55.0) |
| 30 days | all patients | 1.00 | 1.68 | 1.52 | 4.34** |
| | TnT ≦ 0.05 ng/mL | 1.00 | 1.00 | 3.12 | 16.1** |
| | TnT > 0.05 ng/mL | 1.00 | 1.33 | 0.88 | 2.45* |
| 6 months | all patients | 1.00 | 2.09 | 2.39* | 5.38** |
| | TnT ≦ 0.05 ng/mL | 1.00 | 2.12 | 4.10 | 15.7** |
| | TnT > 0.05 ng/mL | 1.00 | 1.84 | 2.31 | 3.92** |
| 12 months | all patients | 1.00 | 1.64 | 1.82 | 3.93** |
| | TnT ≦ 0.05 ng/mL | 1.00 | 4.30 | 7.95* | 24.98** |
| | TnT > 0.05 ng/mL | 1.00 | 1.62 | 1.64 | 2.10* |

*p < 0.05
**p < 0.01

### Example 5

This example demonstrates that measurement of changes in concentration the 53 Kd form of Factor XIIa provides a prediction of risk of secondary myocardial infarction in patients admitted to hospital with myocardial infarction.

Data was obtained on 315 patients admitted to the hospital. Blood samples were obtained at admission and 4 days after admission. Each patient had Factor XIIa measured using assays preferentially measuring the 53 kD form of Factor XIIa. Data from these assays were studied to ascertain if changes in the concentration of the 53 kD form of Factor XIIa provided prediction of the primary clinical endpoints of a second myocardial infarction within 30 days of admission. At 30 days follow-up, 24 patients had suffered a secondary myocardial infarction.

The 53 Kd form of XIIa was measured using high performance liquid chromatography following reaction of the sample with Iodine 125 labelled antibody.

Fab antibody fragments of antibody 2/215 were prepared using an “Immunopure Fab Preparation Kit” (Pierce, 3747 N Meridian Road, PO Box 117, Rockford, Ill. 61105, U.S.A.) according to manufacturer's instructions. These Fab fragments were then radiolabelled with Iodine 125 by Amersham Pharmacia Biotech (Pollards Wood, Nightingales Lane, Chalfont St Giles, HP8 4SP United Kingdom).

1 μL of radiolabelled antibody was added to 1 ml of plasma from each of a number of healthy volunteers. After incubation for 4 hours, the components of the plasma were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min-1 and the injection volume was 100 μl.

The HPLC eluant was monitored by measuring the absorbance at 280 nm, and by monitoring radioactivity using a Flow-Count Radiochromatography detector (LabLogic, Sheffield, UK)

Molecular weight standards were run, and from comparison with these the 53 kD XIIa peak could be identified. Integration of the area under this peak (radioactivity signal) provided a quantitative measure of the 53 kD form of XIIa. Calibration of quantitation was obtained by running standards with known quantities of the 30 kD form of XIIa (βXIIa).

The prognostic utility of the assays was determined by ranking the change in 53 kD Factor XIIa values (from lowest to highest) and then splitting the population into quartiles i.e. the 25% of individuals with the greatest decrease in 53 kD Factor XIIa concentrations between admission and day 4 were in the 1st quartile, whilst the 25% of individuals with the greatest increase in concentrations were in the 4th quartile.

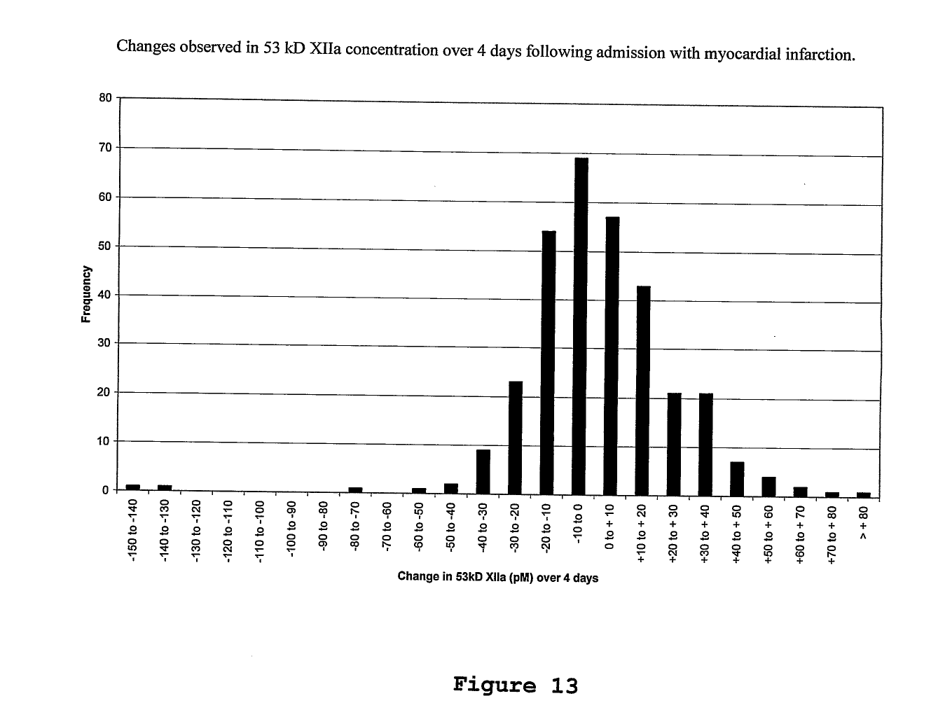

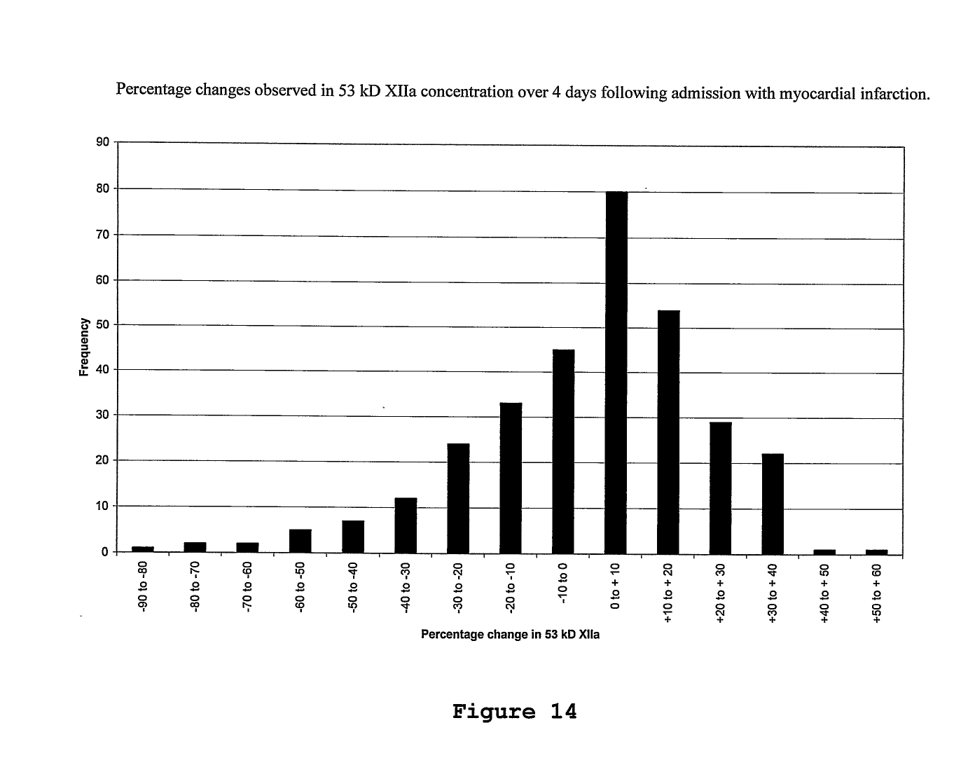

The distribution of changes in the concentration of the 53 kD form of XIIa (expressed as pM) are shown in FIG. 13, and the relative changes in the concentration of the 53 kD form of XIIa (expressed as percentage change relative to the admission value) are shown in FIG. 14.

Event-rates according to change in 53 kD XIIa concentration are given in table II. Both absolute and relative (percentage change from admission) changes in 53 kD XIIa concentration were strongly associated with risk. The odds ratio for recurrent TnT positive events in Q4 as compared to Q1 of change in XIIaA concentration was 15.36 (p=0.0046) for absolute change and 13.97 (p=0.0062) for percentage change relative to the admission value. Therefore, it is concluded that changes in the 53 kD form of XIIa concentration from admission to day 4 after myocardial infarction strongly predict myocardial infarction during 30 days follow-up.

TABLE II
Incidence of TnT positive cardiac events within 30 days
following hospitalisation for MI, related to change in
53 kD XIIa between admission and 4 days post MI.

| | Q1 | Q2 | Q3 | Q4 |
|---|---|---|---|---|
| Change in (pM) | | | | |
| Recurrent TnT + | 1 | 4 | 6 | 13 |
| events (n) | | | | |
| OR (p) | 1.0 | 4.16 (0.104) | 6.41 (0.044) | 15.36 (0.0046) |
| Change in 53 kD XIIa | | | | |
| (expressed as % of | | | | |
| admission value) | | | | |
| Recurrent TnT + | 1 | 4 | 7 | 12 |
| events (n) | | | | |
| OR (p) | 1.0 | 4.16 (0.104) | 7.58 (0.030) | 13.97 (0.0062) |

1. A 53 Kd form of Factor XIIa.

2. A 53 Kd form of Factor XIIa as claimed in claim 1 which is a 53 Kd form of human Factor XIIa having peptides with substantially the amino acid sequences of SEQ ID NO:1 and SEQ ID NO:2.

3. (canceled)

4. An isolated nucleic acid molecule that encodes either one or both of the peptides of the 53 Kd form of Factor XIIa having peptides with substantially the amino acid sequences shown respectively in SEQ ID NO:1 and SEQ ID NO:2.

5. (canceled)

6. A monoclonal or polyclonal antibody that binds to one or more epitopes of a 53 Kd form of Factor XIIa, or an epitope-binding fragment or derivative of said antibody, wherein said antibody has a corrected cross reactivity with one or both of Factor αXIIa and Factor βXIIa of 10% or less.

7. An antibody, fragment or derivative as claimed in claim 6 which is immobilized on a solid support or which is provided with a detectable label.

8. (canceled)

9. A hybridoma cell line that produces a monoclonal antibody as defined in claim 6.

10. A method of producing a monoclonal antibody as defined in claim 6, comprising cultivating a hybridoma cell line capable of producing the antibody in a growth medium and obtaining the antibody from the growth medium.

11. A method of producing a polyclonal antibody as defined in claim 6, comprising inoculating a mammal with an antigen of a 53 Kd form of factor XIIa and purifying an antibody from the serum of said mammal.

12. (canceled)

13. (canceled)

14. A method of detecting and/or determining a 53 Kd form of Factor XIIa in a sample, which comprises subjecting the sample to a qualitative or quantitative immunoassay which comprises the interaction between an antigen and an antibody and the detection and/or determination of any resultant antibody-antigen complex, characterized in that the antibody is an antibody as claimed in claim 6.

15. A method for detecting or determining a 53 Kd form of Factor XIIa in a sample, which comprises carrying out a procedure that is capable of detecting or determining the chosen 53 Kd form of Factor XIIa in preference to other forms of Factor XIIa.

16-24. (canceled)

25. A method as claimed in claim 14 or 15, wherein the sample is a sample of a body fluid or body tissue.

26. A method as claimed in claim 25, wherein the body fluid is blood, plasma, serum, urine, cerebrospinal fluid, saliva, or tears.

27. (canceled)

28. A method as claimed in claim 14 or 15, wherein the 53 Kd form of Factor XIIa under investigation is cellular 53 Kd Factor XIIa.

29-31. (canceled)

32. A method as claimed in claim 14 or 15, wherein the 53 Kd form of Factor XIIa under investigation is lipid bound 53 Kd Factor XIIa.

33-60. (canceled)

61. A method as claimed claim 14 or 15, wherein the sample has been obtained from a subject having a disease or disorder, undergoing a disease or disorder, or after having had a disease or disorder or treatment for the disease or disorder.

62. A method as claimed in claim 61, wherein the disease or disorder involves the coagulation system.

63. A method as claimed in claim 61, wherein the disease or disorder involves haemocoagulation, fibrinolysis, kininogensis, complement activation or angiogenesis, maintaining vascular wholeness and blood pressure, maintaining the constitutive anticoagulant character of the intravascular space, or tissue defence and repair.

64. A method as claimed in claim 61, wherein the disease or disorder is or involves acute or chronic inflammation, shock of any aetiology including septic shock, diabetes, allergy, a thrombo-haemorrhagic disorder, sepsis, spontaneous abortion or an oncological disease.

65. A method as claimed in claim 61, wherein the disease or disorder is or involves intravascular blood coagulation or thromboembolism, a myocardial infarction, acute coronary syndrome or angina.

66. A method as claimed in claim 61, wherein the disease or disorder is or involves thrombosis or stenosis.

67. A method as claimed in claim 61, wherein the disease or disorder is or involves suspected myocardial infarction or acute coronary syndrome.

68. A method as claimed in claim 61, wherein the disease or disorder is or involves sepsis.

69. A method as claimed in claim 61, wherein treatment involves administration of a therapeutic agent and/or involves a surgical procedure.

70. A method as claimed in claim 69, wherein the treatment is coronary artery angioplasty or thrombolysis.

71. A method as claimed in claim 14 or 15, wherein a series of samples obtained from a subject having a disease or disorder, undergoing treatment for a disease or disorder, or after having had a disease or disorder or treatment for the disease or disorder are tested, wherein said samples are obtained during the course of the disease or disorder, during treatment of the disease or disorder, or before treatment is started and/or after treatment has finished.

72-73. (canceled)

74. A method for diagnosing, monitoring, or predicting the susceptibility to, progress of, or outcome of a disease or disorder, or of treatment of the disease or disorder in a subject having or suspected of having the disease or disorder, which comprises detecting or determining a 53 Kd form of Factor XIIa in preference to other forms of Factor XIIa in a sample obtained from the subject, and comparing the results obtained for the subject with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

75. (canceled)

76. A method as claimed in claim 74, wherein the disease or disorder is having had a disease or disorder or treatment for the disease or disorder, wherein the disease or disorder involves the coagulation system, haemocoagulation, fibronolysis, kininogensis, complement activation or angiogenesis, maintaining vascular wholeness and blood pressure, maintaining the constitutive anticoagulant character of the intravascular space, or tissue defense and repair; or wherein the disease or disorder is or involves acute or chronic inflammation, shock of any aetiology including septic shock, diabetes, allergy, a thrombo-haemorrhagic disorder, sepsis, spontaneous abortion or an oncological disease; or wherein the disease or disorder is or involves intravascular blood coagulation or thromboembolism, a myocardial infarction, acute coronary syndrome or angina; or wherein the disease or disorder is or involves thrombosis or stenosis; or wherein the disease or disorder is or involves suspected myocardial infarction or acute coronary syndrome; or wherein the disease or disorder is or involves sepsis.

77. A method as claimed in claim 74, wherein said treatment involves administration of a therapeutic agent and/or involves a surgical procedure; or wherein said treatment is coronary artery angioplasty or thrombolysis.

78. (canceled)

79. A method as claimed in claim 74, wherein samples are obtained upon or following admission of the subject to hospital with suspected myocardial infarction, and wherein low levels of particular forms of 53 Kd Factor XIIa are associated with an increased risk of a secondary troponin positive event.

80. A method as claimed in claim 74, wherein samples are obtained upon or following admission of the subject to hospital with suspected myocardial infarction, and wherein high levels of particular forms of 53 Kd Factor XIIa are associated with an increased risk of a secondary troponin positive event.

81. A method as claimed in claim 74, wherein samples are obtained upon or following admission of the subject to hospital with suspected myocardial infarction, and wherein low levels of particular forms of 53 Kd Factor XIIa are associated with an increased risk of death.

82. A method as claimed in claim 74, wherein samples are obtained upon or following admission to hospital with suspected myocardial infarction, and wherein high levels of particular forms of 53 Kd Factor XIIa are associated with an increased risk of death.

83. A method as claimed in claim 74, wherein samples are obtained upon or following admission to hospital with acute coronary syndrome, and wherein high levels of particular forms of 53 Kd Factor XIIa are associated with an increased risk of death.

84. A method as claimed in claim 74, wherein samples are obtained upon or following admission to hospital with Troponin T (TnT) levels of greater than 0.05 ng/ml, and wherein high levels of particular forms of 53 Kd Factor XIIa are associated with an increased risk of death.

85. A method as claimed in claim 74, wherein samples are obtained upon or following admission to hospital with suspected myocardial infarction, and wherein high levels of particular forms of 53 Kd Factor XIIa are associated with an increased risk of secondary myocardial infarction.

86. A method as claimed in claim 74, wherein high levels of particular forms of 53 Kd Factor XIIa are associated with sepsis.

87. A method comprising carrying out a series of assays for 53 Kd Factor XIIa on samples obtained from subjects having a disease or disorder or treatment for a disease or disorder, and selecting an assay that provides information on 53 Kd Factor XIIa levels that is relevant to the disease or disorder or the treatment.

88. A method for providing an assay for 53 Kd Factor XIIa suitable for providing information relevant for diagnosing, monitoring, or predicting the susceptibility to, progress of, or outcome of a disease or disorder, or of treatment of the disease or disorder in a subject having or suspected of having the disease or disorder, which comprises carrying out a series of assays for 53 Kd Factor XIIa on samples obtained from subjects having the disease or disorder or the treatment, and determining which assay(s) provide information on 53 Kd Factor XIIa levels that is relevant to diagnosing, monitoring, or predicting the susceptibility to, progress of, or outcome of the disease or disorder, or of treatment of the disease or disorder.

89. A method as claimed in claim 88, comprising comparing the results obtained for 53 Kd Factor XIIa in the samples obtained from subjects having the disease or disorder or the treatment with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

90. (canceled)

91. A method as claimed in claim 88 or 89, wherein the disease or disorder involves the coagulation system, haemocoagulation, fibrinolysis, kininogensis, complement activation or angiogenesis, maintaining vascular wholeness and blood pressure, maintaining the constitutive anticoagulant character of the intravascular space, or tissue defence and repair; or wherein the disease or disorder is or involves acute or chronic inflammation, shock of any aetiology including septic shock, diabetes, allergy, a thrombo-haemorrhagic disorder, sepsis, spontaneous abortion or an oncological disease; or wherein the disease or disorder is or involves intravascular blood coagulation or thromboembolism, a myocardial infarction, acute coronary syndrome or angina; or wherein the disease or disorder is or involves thrombosis or stenosis; or wherein the disease or disorder is or involves suspected myocardial infarction or acute coronary syndrome; or wherein the disease or disorder is or involves sepsis.

92. A method as claimed in claim 88 or 89, wherein treatment involves administration of a therapeutic agent and/or involves a surgical procedure; or wherein said treatment is coronary artery angioplasty or thrombolysis.

93. A method as claimed in claim 88 or 89, wherein the samples are obtained upon or following admission of the subject to hospital with suspected myocardial infarction.

94. (canceled)

95. A database comprising the results obtained according to a method as claimed in claim 88.

96. A method comprising detecting or determining 53 Kd Factor XIIa in preference to other molecular weight forms of Factor XIIa in a sample from a subject, characterised in that the sample is a sample if urine.

97. A method for diagnosing or monitoring a disease or disorder, or monitoring treatment of the disease or disorder, which comprises detecting or determining Factor 53 Kd XIIa in preference to other molecular weight forms of Factor XIIa in the urine of a subject having or suspected of having the disease or disorder, wherein the disease is or involves renal function, renal disease or renal damage, or treatment therefore.

98. (canceled)

99. A method as claimed in claim 96 or 97, wherein the results obtained for the subject are compared with the results obtained using the same assay for samples obtained from at least any one or more of the following:
(i) subjects having the disease or disorder, for example, impaired renal function, renal disease or renal damage;
(ii) subjects having the disease or disorder, for example impaired renal function, renal disease or renal damage, which subjects were monitored in relation to the progress and/or outcome of the disease or disorder, for example impaired renal function, renal disease or renal damage;
(iii) subjects having the disease or disorder, for example impaired renal function, renal disease or renal damage and having the treatment therefor;
(iv) subjects having the disease or disorder, for example impaired renal function, renal disease or renal damage and the treatment, which subjects were monitored in relation to the treatment in relation to the progress and/or outcome of the disease or disorder, for example impaired renal function, renal disease or renal damage;
(v) subjects who do not have the disease or disorder, for example impaired renal function, renal disease or renal damage;
(vi) the same subject before the onset of the disease or disorder, for example impaired renal function, renal disease or renal damage or before the start of the treatment of the disease or disorder, for example impaired renal function, renal disease or renal damage; and
(vii) the same subject at an earlier or later stage of the disease or disorder, for example impaired renal function, renal disease or renal damage or the treatment, or before the onset of the disease or disorder, for example impaired renal function, renal disease or renal damage.

100. (canceled)


SEQUENCE LISTING (2 sequences)

SEQ ID NO: 1 - protein, 226 aa, Homo sapiens
KCFEPQLLRFFHKNEIWYRTEQAAVARCQCKGPDAHCQRLASQANPCLHGGRCLEVEGHRLCHCPVGYTGPFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQR

SEQ ID NO: 2 - protein, 243 aa, Homo sapiens
VVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS